SEQUENCE LISTING (4 sequences)

SEQ ID NO: 1 - protein, 506 aa, Caenorhabditis elegans
MTSPSSDEDIIDIRVIKEEPESEPDSEAEPATTTNSTDSEDSVEQENKKLLETEKNRKREQKHKMLPNGTTSGTSDTGNQVPATSSAASSVDYTAMNAQDYLPTYSNTTLNYQPYQYQTAANGLLNYNNYSQYATANQLGSNYISPANFMQGGGISPLGFTTGTTGATTAAASVATSSASAVIGRSNGRSSSTVAASPADRSYSGVSGGQGQELTIQEFETVTEKIRRHGTYGQSKPPYSYISLITMAIQKSNSRQLTLSEIYNWIMDLFPYYQNNQQRWQNSIRHSLSFNDCFVKVARSPDKPGKGSFWTLHEHCGNMFENGCYLRRQKRFKVKEREPSRKKRNANSQQLHQQQHIPKMEIKEEDPTSITTTSSLGAYSLIPQISTKKEIKEELKAVQDATAAAANLGLIDPSGTPSAVNHSQPTSVISSVGTLGTTQAQMTLNGQYASPYLYSSDFATILPQSQNFLNNTLYNTTSSYPGIDYTNGVYQNTLYSSTNPNSAANL

SEQ ID NO: 2 - protein, 472 aa, Homo sapiens
MLGTVKMEGHETSDWNSYYADTQEAYSSVPVSNMNSGLGSMNSMNTYMTMNTMTTSGNMTPASFNMSYANPGLGAGLSPGAVAGMPGGSAGAMNSMTAAGVTAMGTALSPSGMGAMGAQQAASMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 3 - protein, 457 aa, Homo sapiens
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 4 - protein, 350 aa, Homo sapiens
MLGSVKMEAHDLAEWSYYPEAGEVYSPVTPVPTMAPLNSYMTLNPLSSPYPPGGLPASPLPSGPLAPPAPAAPLGPTFPGLGVSGGSSSSGYGAPGPGLVHGKEMPKGYRRPLAHAKPPYSYISLITMAIQQAPGKMLTLSEIYQWIMDLFPYYRENQQRWQNSIRHSLSFNDCFVKVARSPDKPGKGSYWALHPSSGNMFENGCYLRRQKRFKLEEKVKKGGSGAATTTRNGTGSAASTTTPAATVTSPPQPPPPAPEPEAQGGEDVGALDCGSPASSTPYFTGLELPGELKLDAPYNFNHPFSINNLMSEQTPAPPKLDVGFGGYGAEGGEPGVYYQGLYSRSLLNAS

(19) United States
(12) Patent Application Publication
(10) Pub. No.: US 2009/0092551 A1
(43) Pub. Date: Apr. 9, 2009

(54) Increasing lifespan by modulation of pha-4

(75) Inventors: Andrew Dillin, La Jolla, CA (US); Hugo Aguilaniu, Lyon (FR); Siler Panowski, San Diego, CA (US); Suzanne C. Wolff, Carlsbad, CA (US)

Correspondence Address: QUINE INTELLECTUAL PROPERTY LAW GROUP, P.C., P O BOX 458, ALAMEDA, CA, 94501, US

(73) Assignee: The Salk Institute for Biological Studies

(21) Appl. No.: 12/218,976

(22) Filed: Jul. 17, 2008

Related U.S. Application Data
(60) Provisional application No. 60/961,434, filed on Jul. 19, 2007.

(51) Int. Cl. A61K 49/00; C12Q 1/68; A61K 39/395

(52) U.S. Cl. 424/9.1; 435/6; 424/130.1

(57) ABSTRACT

The roles of the pha-4 and daf-16 genes in diet-restricted induced longevity are described and characterized. Pha-4 acts, e.g., in the absence of daf-16, to increase lifespan, e.g., in nematodes. Given the role that pha-4 and daf-16 play in the mediation of longevity, they represent targets for modulation of life span. Methods of increasing life span and delaying age onset diseases by modulation of pha-4 activity are disclosed, as are screening methods for identifying compounds that modulate pha-4 and/or daf-16 activity. In addition, recombinant animals expressing the pha-4 gene and not the daf-16 gene, and methods of using the pha-4 and/or daf-16 genes to modulate longevity and age-onset diseases are described.

### CROSS REFERENCE TO RELATED APPLICATIONS

This application claims priority to and benefit of U.S. Ser. No. 60/961,434 filed Jul. 19, 2007 by Dillin et al., Entitled “Increasing Lifespan by Modulation of pha-4.” This prior application is incorporated herein by reference in its entirety for all purposes.

### FIELD OF THE INVENTION

The current invention relates to the field of longevity enhancement. More specifically, the present invention provides methods for increasing lifespan, e.g., by modulating pha-4 and/or daf-16 expression, as well as screening methods for identifying compounds that modulate pha-4 and/or daf-16, thereby modulating longevity.

### BACKGROUND OF THE INVENTION

Aging, e.g., in mammals or other animals, can have profound negative effects on the cognitive and motor functions of the subject. Genes that regulate the aging pathways and genes that could slow, pause, or decrease the effects of aging and/or increase lifespan are of great interest, both because of their potential to increase longevity and/or enhance quality of life during the later part of one's lifespan. However, there are many pathways that regulate aging and the genes that control them and the connections between them are poorly understood. (See, e.g., Clancy et al, Dietary restriction in long-lived dwarf flies. Science 296, 319 (2002); Clancy et al. Extension of life-span by loss of CHICO, a Drosophila insulin receptor substrate protein. Science 292, 104-106 (2001); Tatar, M. et al. A mutant Drosophila insulin receptor homolog that extends lifespan and impairs neuroendocrine function. Science 292, 107-110 (2001); and Tu, M. P., Epstein, D. & Tatar, M. The demography of slow aging in male and female Drosophila mutant for the insulin-receptor substrate homologue chico. Aging Cell 1, 75-80 (2002)).

For example, reduced food intake as a result of dietary restriction increases the lifespan of a wide variety of metazoans and delays the onset of multiple age-related pathologies. This is a conserved phenomenon in a number of species, e.g., yeast, worms, flies, mice, waterstriders, guppies, chickens, labradors, and rats. Dietary restriction elicits a genetically programmed response to nutrient availability that cannot be explained by a simple reduction in metabolism or slower growth of the organism.

The insulin/IGF-1 signaling (IIS) pathway is a key regulator of the aging process in worms, flies and mice, but its role in the regulation of diet-restriction-mediated longevity remains ambiguous. Perfunctorily, it seems probable that the regulation of nutrient homeostasis and ageing by the IIS pathway might overlap with any regulatory networks affected by dietary restriction. However, prior research in worms suggests that diet-restriction-mediated increases in longevity can occur independently of the forkhead boxO (FOXO) transcription factor DAF-16 (See, Houthoofd, K., et al. Life extension via dietary restriction is independent of the Ins/IGF1 signalling pathway in Caenorhabditis elegans. Exp. Gerontol. 38, 947-954 (2003); Lakowski & Hekimi. The genetics of caloric restriction in Caenorhabditis elegans. Proc. Natl. Acad. Sci. USA 95, 13091-13096 (1998)), whereas the extended longevity of all known IIS mutants is completely dependent on DAF-16 (See, Kenyon, et al., C. elegans mutant that lives twice as long as wild type. Nature 366, 461-464 (1993); Henderson & Johnson, daf-16 integrates developmental and environmental inputs to mediate aging in the nematode Caenorhabditis elegans. Curr. Biol. 11, 1975-1980 (2001); Lin, K et al. daf-16: An HNF-3/forkhead family member that can function to double the life-span of Caenorhabditis elegans. Science 278, 1319-1322 (1997); Lin, K et al., Regulation of the Caenorhabditis elegans longevity protein DAF-16 by insulin/IGF-1 and germline signaling. Nature Genet. 28, 139-145 (2001); Ogg, S. et al. The Fork head transcription factor DAF-16 transduces insulin-like metabolic and longevity signals in C. elegans. Nature 389, 994-999 (1997)); thus it seems unlikely that reduced food intake simply elicits an environment of reduced insulin signaling. From this hypothesis, it was initially predicted that genetic components would not be shared across these two pathways.

Genetically, smk-1 is an essential co-regulator of the longevity function of daf-16, and previous data suggested a relationship in which these two genes cannot affect lifespan independently of each other (See, Wolff, S. et al. SMK-1, an essential regulator of DAF-16-mediated longevity. Cell 124, 1039-1053 (2006). Daf-16 is dispensable for the long lifespan of eat-2(ad1116) mutant animals (a genetic surrogate of dietary restriction exhibiting a reduced rate of pharyngeal pumping representative of eating). See, Lakowski & Hekimi, Proc. Natl. Acad. Sci. USA 95, 13091-13096 (1998); Avery, L. The genetics of feeding in Caenorhabditis elegans. Genetics 133, 897-917 (1993). Thus, it was surprising to find that smk-1 was required for the extended lifespan of eat-2(ad1116) mutant animals (see, e.g., Methods, FIG. 1A and the table in FIG. 12). These data elicited the hypothesis that under conditions of low nutrient signaling, smk-1 could interact genetically with a forkhead-like transcription factor other than daf-16 to mediate the transcriptional response to dietary restriction. Therefore, more information is needed to determine the role of the various forkhead transcription factors in the aging process.

In the nematode worm Caenorhabditis elegans (an important model system for the study of aging due to its short lifespan and amenability to genetic and molecular analysis), the forkhead transcription factor PHA-4 has an essential role in the embryonic development of the foregut and is orthologous to genes encoding the mammalian family of Foxa transcription factors, including Foxa1, Foxa2 and Foxa3. For example, foxa1, also referred to as hepatocyte nuclear factor 3α or HNF3α, is a forkhead DNA binding protein that is orthologous to pha-4. Foxa family members have important roles during development, but also act later in life to regulate glucagon production and glucose homeostasis, particularly in response to fasting. Determination of the role of these transcription factors in aging and longevity would lead to novel methods of screening for modulators of longevity, as well as novel methods of modulating lifespan.

Thus, there is a continuing need for more information to determine what genes are involved in regulating dietary restriction induced longevity and more information regarding the pathways and possible connections between the insulin/IGF-1 signaling and dietary restriction. The current invention provides these and other benefits which will be apparent upon examination of the current specification, claims, and figures.

### SUMMARY OF THE INVENTION

Pha-4 and daf-16 are shown herein to mediate diet-restriction induced longevity. The connection between these genes and longevity is used to provide screening methods, e.g., whole organism and cell-based methods, for identifying compounds that modulate longevity and delay age-onset diseases and conditions. In addition, methods are presented herein for using these two genes to modulate longevity in an animal and for delaying age onset diseases.

In one embodiment, methods of screening for a longevity modulator are provided. In one aspect, the methods comprise providing a non-human animal that expresses pha-4 or a homolog thereof and exhibits reduced expression of daf-16 or a homolog thereof. The animal is administered a test compound, e.g., a potential modulator, such as an antibody, a protein, a small molecule, an antisense molecule, a nucleic acid, or the like. After administration of a test compound, the animal is monitored or assayed to detect any changes in a pha-4 parameter, e.g., as compared to an animal that has not been administered the compound. A change in any pha-4 parameter indicates that the test compound modulates longevity. Typical pha-4 parameters comprise lifespan or an activity or expression level of pha-4, sod-1, sod-2, sod-4, sod-5, daf-16 or any homologs thereof. For example, an increased lifespan or an increase in expression of pha-4 or a homolog thereof indicates that the test compound is optionally used to increase longevity in the animal.

Animals that are optionally used for the screening methods of the invention include, but are not limited to, nematodes, e.g., C. elegans, mice, flies, e.g., drosophila, and the like. In mammals, the pha-4 homolog is typically a foxa gene, e.g., foxa1, foxa2, or foxa3. In this example, lifespan of the animal and/or expression of a foxa gene is assayed and an increase or decrease in either is considered an indication that the test compound is a modulator of longevity.

In one aspect, the non-human animal is an adult nematode, such as C. elegans and administering the modulator to the non-human animal comprises feeding the modulator to the non-human animal. The animal is also optionally subjected to dietary restriction. The animals used in the screening methods typically exhibit reduced expression of daf-16 or do not express daf-16 at all, e.g., the animals are transgenic animals with a knock out version of daf-16.

In another embodiment, cell based assays are provided for identifying modulators of longevity, e.g., through modulation of pha-4 and/or daf-16. The cell-based methods typically comprise contacting a cell that expresses pha-4 or a homolog thereof with a test agent. The cells also typically exhibit reduced expression of daf-16 or a homolog thereof. The cells are then monitored or assayed for a pha-4 parameter in the cell, wherein a change in the pha-4 parameter relative to a control sample without the test agent identifies the compound that modulates longevity. The pha-4 parameters that are typically monitored include, but are not limited to, activity or expression levels of pha-4, sod-1, sod-2, sod-4, sod-5, daf-16, or any homologs thereof. For example, an increase in the expression level of pha-4 is an indication that the test compound increases longevity. Typical test agents include, but are not limited to, antibodies, proteins, small molecules, antisense molecules, nucleic acids (e.g., DNA, or RNA), and the like.

In another embodiment, a system for screening for compounds that modulate longevity is provided. The system typically comprises an array of non-human animals in containers. The non-human animals are typically nematodes, such as adult C. elegans, or flies that express pha-4 or a homolog thereof. In addition, the animals optionally express daf-16 or a homolog thereof, although the animals optionally have reduced expression of daf-16 or do not express daf-16 at all. The animals are optionally subjected to dietary restriction. The system also comprises a monitoring module that monitors a pha-4 parameter of the non-human animals in the array following administration of a test compound, e.g., as described above. The pha-4 parameter is optionally lifespan or activity or expression of pha-4, sod-1, sod-2, sod-4, sod-5, and/or daf-16 or any homologs thereof. The pha-4 factor is also optionally a combination of two or more of the above factors. For example, in an animal that expresses both pha-4 and daf-16, expression levels of both genes is optionally monitored, wherein the presence of both an increase in pha-4 expression and a decrease in daf-16 is indicative of longevity modulation by the test compound. The system further comprises a correlation module, e.g., a computer comprising software, for correlating any changes in pha-4 parameters to changes in longevity, thereby identifying the compounds that modulate longevity.

In another embodiment, a method of identifying a modulator of longevity, e.g., by monitoring both pha-4 expression and daf-16 expression is provided. In one aspect, the screening assay is a cell-based assay and in another assay, the method is a whole organism assay.

In whole organism screening assays, the methods comprise providing a non-human animal, which animal expresses pha-4 or a homolog thereof and daf-16 or a homolog thereof. The methods further comprise administering a test compound to the non-human animal; and, monitoring expression of pha-4 or the homolog thereof and expression of daf-16 or the homolog thereof in the non-human animal. For example, an increase in pha-4 expression and a decrease in daf-16 expression indicate that the test compound modulates longevity. A typical animal used for screening is a nematode, e.g., C. elegans, Typically, the animal used for screening is an adult animal, e.g., an adult C. elegans, which is fed the test compound. In some embodiments, the animal is subjected to dietary restriction.

In cell-based assays, methods of identifying a modulator of longevity typically comprise providing a cell that expresses pha-4 or a homolog thereof and daf-16 or a homolog thereof. The cell is contacted with a test compound; and, typically two or more pha-4 parameters are monitored in the cell. For example, pha-4 expression and daf-16 expression are monitored in the cell. An increase in expression of pha-4 or the homolog thereof and a decrease in expression of daf-16 or the homolog thereof indicate that the test compound modulates longevity.

In another aspect, the present invention provides a method of increasing longevity in an animal. The method comprises administering a compound that increases expression of pha-4 or a homolog thereof to the animal. In some embodiments, the compound administered to the animal to increase longevity also decreases expression of daf-16 or a homolog thereof. In other embodiments, a second compound is administered to the animal, e.g., to decrease expression of daf-16 or a homolog thereof.

In another aspect, the invention provides a method of delaying onset of an age-related disease in an animal. The method typically comprises modulating, e.g., increasing, expression of pha-4 or a homolog thereof in the animal. The expression of daf-16 or a homolog thereof is also optionally modulated, e.g., decreased, to delay onset of age-related diseases. The method of delaying onset of age-related diseases and conditions typically comprises administering a longevity modulator to the subject, e.g., animal or human. For example, the animal is optionally administered a compound that modulates, e.g., increases expression of, pha-4 or a homolog thereof, such as a modulator identified by any of the assays presented herein. The modulator also optionally modulates expression of daf-16. For example, a modulator that increases expression of pha-4 and decreases expression of daf-16 can be administered to an animal to delay onset of age-related diseases and/or conditions. An animal or patient being treated to delay onset of disease or to extend longevity is optionally subjected to dietary restriction in addition to being administered a modulator of the invention.

In another aspect, a method for modulating longevity of an animal is provided. For example, modulating, e.g., increasing or decreasing, expression of pha-4 or a homolog thereof is one method provided herein. In one embodiment, the method also optionally includes modulating, e.g., decreasing, expression of daf-16 or a homolog thereof in the animal. Modulating longevity typically comprises administering to the animal a longevity modulator that affects pha-4 or the homolog thereof and/or daf-16 or a homolog thereof in the animal. For example, increasing expression of pha-4 using a modulator of the invention is one embodiment of the invention. Alternatively, a compound that increases expression of pha-4 or the homolog thereof and decreases the expression of daf-16 or a homolog thereof is administered to an animal or subject, e.g., a human, desiring longevity modulation. In some embodiments, the patient or subject is subjected to dietary restriction in addition to modulation of pha-4 expression.

In another aspect, the invention provides a method of increasing longevity or delaying onset of an age-related disease in a subject, comprising administering to the subject, an agent identified by any of the methods described herein, e.g., cell-based, non-cell-based, or whole organism screening methods. In another aspect modulators of longevity are provided, such as modulators of pha-4 and/or daf-16 expression or activity.

In both cell based assays and whole organism assays, a test compound of the invention typically comprises an antibody, a protein, a small molecule, an antisense molecule, a nucleic acid, e.g., DNA or RNA, or the like. Similarly, the modulators of the invention are optionally antibodies, proteins, small molecules, antisense molecules, nucleic acids, e.g., DNA or RNA, or the like. Additionally, for any of the methods or treatments described herein, a pha-4 homolog optionally comprises a foxa gene, such as foxa1, foxa2, and/or foxa3.

In another aspect, the inventions provides modulators of longevity identified using the above methods and methods of increasing longevity and/or delaying onset of age-related diseases that use modulators identified using the methods described herein.

These and other features of the invention will become more fully apparent when the following detailed description is read in conjunction with the accompanying figures and claims.

### BRIEF DESCRIPTION OF THE DRAWINGS

FIGS. 1A-1F provide data showing that smk-1 and pha-4 are required for diet-restriction-mediated longevity. Black lines indicate N2 wild-type worms grown on empty vector RNAi bacteria unless noted. Each figure is described in more detail below.

FIG. 1A: eat-2(ad1116) worms fed empty vector RNAi bacteria (green line) lived significantly longer than eat-2(ad1116) worms fed smk-1 RNAi bacteria (light blue line).

FIG. 1B: Dietary restriction using non-RNAi bacterial dilution (BDR) results in a parabolic curve for wild-type worms (black line) and daf-16(mu86)-null mutant animals (blue line), but not pha-4(zu225); smg-1 (cc546ts) temperature sensitive (ts) mutant animals (red line). The inset shows a lifespan plot of wild-type (N2; black line) and pha-4(zu225); smg-1(cc546ts) (P4; red line) worms with 7.5×10⁸ cells ml⁻¹ (ad libitum; AL) or 7.5×10⁷ cells ml⁻¹ (dietary restriction; DR). Error bars, s.e.m.

FIG. 1C: eat-2(ad1116) mutant animals fed pha-4 RNAi bacteria from the L1 larval stage (red line) were shorter lived than animals fed vector RNAi bacteria (green line). This shows that pha-4 is required for dietary restriction induced longevity.

FIG. 1D: daf-2(e1368) mutant animals fed either vector RNAi bacteria (green line) or pha-4 RNAi bacteria (red line) lived significantly longer than when fed daf-16 RNAi bacteria (blue line). This shows that pha-4 is not required for long lifespan due to reduced insulin/IGF-1 signaling (as provided by the daf-2 mutants).

FIG. 1E: Wild-type (N2) animals fed 50% cyc-1 and 50% vector RNAi bacteria (green line) or 50% cyc-1 and 50% pha-4 RNAi bacteria (red line) showed a similar lifespan extension compared to N2 animals fed vector RNAi bacteria alone (black line). N2 animals fed 50% pha-4 and 50% vector RNAi bacteria (yellow line) had a slightly shorter lifespan. Lifespan analyses of animals with reduced mitochondrial electron transport chain were performed at 15° C. This indicates that pha-4 is not required for increased longevity due to reduced mitochondrial electron transport chain activity.

FIG. 1F: Wild-type worms fed pha-4 RNAi bacteria starting from either day 1 of adulthood (red line) or the L1 larval stage (orange line) lived significantly shorter than worms fed vector RNAi bacteria (black line).

FIG. 2: Shows that pha-4 is required during adulthood to regulate longevity in response to dietary restriction. Eat-2(ad1116) mutants were transferred to pha-4 RNAi bacteria at the L4 larval stage (blue line; mean lifespan 18±0.5 days; mean±s.e.m.) or day 1 of adulthood (red line; mean lifespan 19.2±0.4 days) and in both cases showed a decreased lifespan compared with eat-2(ad1116) animals fed vector RNAi bacteria (green line; mean lifespan 23.8±0.6 days). Mean lifespan of N2 worms fed vector RNAi bacteria (black line) was 18.1±0.4 days.

FIGS. 3A-3D illustrate the regulation and localization of pha-4 in response to dietary restriction.

FIG. 3A: A pha-4-rfp transcriptional fusion construct (see Examples and Methods Summary) reveals pha-4 expression in the intestine and somatic gonad (spermatheca, black arrow) of the adult worm.

FIG. 3B shows that AD84 worms reveal PHA-4-GFP nuclear localization (Panel i) in intestinal cells (Panels ii and iii), head neurons (Panels iv and v), and tail neurons (Panels vi and vii).

FIG. 3C: AD84 worms carrying the pha-4-gfp transgene were placed in high-food cultures (7.5×10⁸ cells ml⁻¹; panels i and ii) or low-food cultures (7.5×10⁷ cells ml⁻¹; panels iii and iv). Nuclear localization of PHA-4-GFP in intestinal nuclei (red arrows) remained constant under both conditions (see Methods). Images were taken on day four of adulthood.

FIG. 3D provides results of semi-quantitative RT-PCR. Panel (i) reveals an increase in pha-4 messenger RNA levels in eat-2(ad1116) animals compared with wild-type animals of ˜80% (unpaired two-tailed t-test P-value<0.001; error bars, s.d.; Panels (ii) and (iii) provide Q-PCR analysis of the pha-4 gene and the pha-4 3′ UTR confirms that pha-4 mRNA levels were increased in eat-2(ad1116) worms (red bars) compared with wild-type worms (black bars) (unpaired two-tailed t-test P-value<0.0001; errors bars, s.d. Note that n=3 for both semi-quantitative RT-PCR and Q-PCR experiments.

FIG. 4 shows that increased dosage of pha-4 extends lifespan. Transgenic daf-16(mu86)-null mutant worms carrying an overexpressor (OE) pha-4 transgene (AD115, green line) were long-lived compared with daf-16(mu86) worms (AD105, black line). This lifespan extension was fully suppressed by pha-4 RNAi (red line). Further statistical data can be found in the table in FIG. 11.

FIGS. 5A-5I show differential transcriptional regulation of sods by pha-4 and daf-16 in response to dietary restriction and IIS. All mRNA expression levels were determined using Q-PCR analysis and performed in parallel as provided in the examples methods sections. Q-PCR reactions were run in quadruplicate and averages from one representative set of reactions are depicted in graphs. Error bars represent s.d. for the reaction depicted and asterisks indicate a change in expression with an unpaired two-tailed t-test P-value <0.005 as compared with black bars of the same graph and gene. For FIGS. 5A, 5C, 5E, and 5G, mRNA levels are of worms fed vector RNAi bacteria.

FIG. 5A: Shows that sod-3 expression levels were increased in daf-2(e1370) animals (blue bar) compared with wild-type N2 (black bar) and eat-2(ad1116) animals (red bar).

FIG. 5B: Shows that sod-3 mRNA expression levels in eat-2(ad1116) mutant worms were unaffected by pha-4 RNAi (red bar), but were decreased in response to daf-16 RNAi (blue bar).

FIG. 5C: Shows that sod-1 mRNA levels were increased in eat-2(ad1116) worms (red bar) compared with wild-type worms (black bar).

FIG. 5D: Shows that sod-1 mRNA expression levels were decreased in eat-2(ad1116) worms fed pha-4 RNAi (red bar).

FIG. 5E: Shows that all sods, except sod-3, were upregulated in eat-2(ad1116) mutant animals (red bars) compared with wild-type animals (black bars).

FIG. 5F: Shows that sod-1, sod-2, sod-4 and sod-5 mRNA expression in eat-2(ad1116) worms was greatly decreased when worms were fed pha-4 RNAi bacteria (red bars) compared with eat-2(ad1116) worms fed vector RNAi bacteria (black bars).

FIG. 5G: Shows daf-2(e1370) mutant worms (blue bars) had elevated levels of sod-1, sod-3 and sod-5 mRNA when compared with wild-type worms (black bars).

FIG. 5H: Shows daf-2(e1370) mutant animals fed daf-16 RNAi bacteria (blue bars) had reduced levels of sod-1, sod-3 and sod-5 compared with daf-2(e1370) mutant animals fed vector RNAi (black bars).

FIG. 5I: Provides a model depicting differential regulation of sods in response to IIS and dietary restriction (DR) mediated by DAF-16 and PHA-4, respectively. Types (Fe/Mn, Cu/Zn) and location, if known, of superoxide dismutases are listed next to the genes (See, Hunter et al., Cloning, expression, and characterization of two manganese superoxide dismutases from Caenorhabditis elegans. J. Biol. Chem. 272, 28652-28659 (1997); Suzuki et al., Cloning, sequencing and mapping of a manganese superoxide dismutase gene of the nematode Caenorhabditis elegans. DNA Res. 3, 171-174 (1996); Fujii et al., A novel superoxide dismutase gene encoding membrane-bound and extracellular isoforms by alternative splicing in Caenorhabditis elegans. DNA Res. 5, 25-30 (1998); Larsen, P. L. Aging and resistance to oxidative damage in Caenorhabditis elegans. Proc. Natl. Acad. Sci. USA 90, 8905-8909 (1993)).

FIG. 6 shows that the foxa family of transcription factors are the closest human orthologs to pha-4. The forkhead domains of the proteins are 91% similar and 85% identical. Alignment of proteins was performed using ClustlW and visualized using BoxShade 3.31. Black shading indicates identical amino acids. Grey shading indicates similar amino acids. Forkhead domains are bracketed by red arrows.

FIG. 7 shows that daf-16 is not required for DR mediated longevity. Daf-16(mu86) null mutant worms responded to dietary restriction by BDR. The mean lifespan, 16.2±0.7 days (LS±SEM), of worms at optimal DR (7.5e7 cells/ml), was significantly longer than the lifespan, 9.0±0.4 days, of worms fed ab libitum (7.5e8 cells/ml). Log-rank (Mantel-Cox) p<0.001. Experiment was performed at 20° C. as described in examples and methods.

FIG. 8 shows that pha-4 RNAi reduces pha-4 expression. pha-4 RNAi used in the pumping rate assays was able to knock down pha-4 levels after forty-eight hours as seen in AD84(pha-4::gfp) worms grown on pha-4 RNAi (panels c and d) compared to worms grown on vector RNAi (panels a and b). Ten worms were examined per RNAi condition and representative images are shown for each condition. Panels a and c consist of DIC and GFP merged images with GFP depicted in green. Panels b and d depict only the GFP channel with GFP shown in white.

FIG. 9 is a table providing data regarding the effect of forkhead genes upon eat-2(ad1116) lifespan.

FIG. 10 is a table providing lifespan data for worms undergoing bacterial dietary restriction.

FIG. 11 is a table providing data on the effect of overexpression of pha-4 on lifespan.

FIG. 12 shows a table of lifespan data corresponding to FIGS. 1A-1F.

FIG. 13 illustrates that smg-1(cc546ts) worms respond to BDR. 5 mg-1 worms under BDR conditions were long-lived when fed the optimal bacterial concentration compared to smg-1 worms fed ab libitum.

FIGS. 14A and 14B show that the long lifespan of daf-2 mutant worms is not suppressed by the loss of pha-4. All lifespan information and data corresponding to these graphs is found in the table in FIG. 12.

FIG. 14A shows that pha-4RNAi does not suppress the long lifespan of daf-2(mu150) mutant animals. The lifespan of daf-2(mu150) mutant worms fed pha-4 RNAi (red line) was not shortened compared to daf-2(mu150) worms fed vector RNAi (green line). daf-2(mu150) mutant worms fed daf-16 RNAi (blue line) had a slightly shorter lifespan than CF512 mutants worms (black line). Lifespans were placed at 25° C. from the L3 larval stage through day 1 of adulthood to block progeny production due to fer-15(b26)II; fem-1(hc17) mutations in the CF512 background. Worms were kept at 20° C. for the remainder of the lifespans.

FIG. 14B shows that pha-4 RNAi does not fully suppress the long lifespan of daf-2(e1370) mutant animals. The lifespan of daf-2(e1370) mutant animals is similar when worms are fed either vector RNAi (green line), or pha-4 RNAi bacteria (red line). daf-16 RNAi bacteria (blue line) suppressed the lifespan of daf-2(e1370) mutants almost back to that of wild type worms fed vector RNAi (black line). The mild, 12.5%, reduction of the mean lifespan of daf-2(e1370) mutant animals may be explained by the fact that the two classes of daf-2 alleles can act very differently, daf-2(e1370) mutant animals exhibit a slight eat mutant phenotype, whereas daf-2(e1368) mutant animals do not, and this may account for the slight decrease we see in daf-2(e1370), but not in daf-2(e1368) or daf-2(mu150).

FIGS. 15A-15C illustrate that the loss of pha-4 does not fully suppress the lifespan of animals with altered ETC. Supplemental data corresponding to these figures is found in FIG. 12.

FIG. 15A: pha-4(zu225), smg-1(cc546ts) mutant worms fed cyc-1 RNAi bacteria (red line) live significantly longer than pha-4(zu225), smg-1(cc546ts) worms fed vector RNAi bacteria (yellow line). The difference in lifespan of pha-4(zu225), smg-1(cc546ts) worms fed cyc-1 RNAi compared to smg-1(cc546ts) fed cyc-1 RNAi (green line) is comparable to the lifespan differences seen between these two strains when both fed vector RNAi bacteria.

FIG. 15B: pha-4 RNAi does not suppress the long lifespan of isp-1(qm130) mutant worms. pha-4 RNAi bacteria shortens lifespan when fed to isp-1(qm130) worms (red line) as compared to isp-1(qm130) worms fed vector RNAi bacteria (green line). This ˜15% decrease is comparable to the decrease in lifespan seen in wild type (N2) worms fed pha-4 RNAi (yellow line) compared to N2 worms fed vector RNAi bacteria (black line).

FIG. 15C: daf-16(mu86) null worms fed vector RNAi (yellow line) or cyc-1 RNAi (blue line) exhibit decreased lifespans when compared to wild type (N2) worms fed either vector RNAi (black line) or cyc-1 RNAi (green line).

### DETAILED DESCRIPTION

The present invention provides a newly discovered, adult-specific function for pha-4 in the regulation of diet-restriction-mediated longevity. Based on this evidence, screening methods for identifying longevity modulators and methods of increasing longevity are provided. For example, methods of screening animals or cells that are administered test compounds and assaying them for changes in a pha-4 related parameter are provided. The present invention also provides evidence that both pha-4 and daf-16 are involved in dietary restricted longevity. Therefore, methods of increasing longevity and delaying onset of age related diseases and conditions using pha-4 or pha-4/daf-16 modulators are also provided.

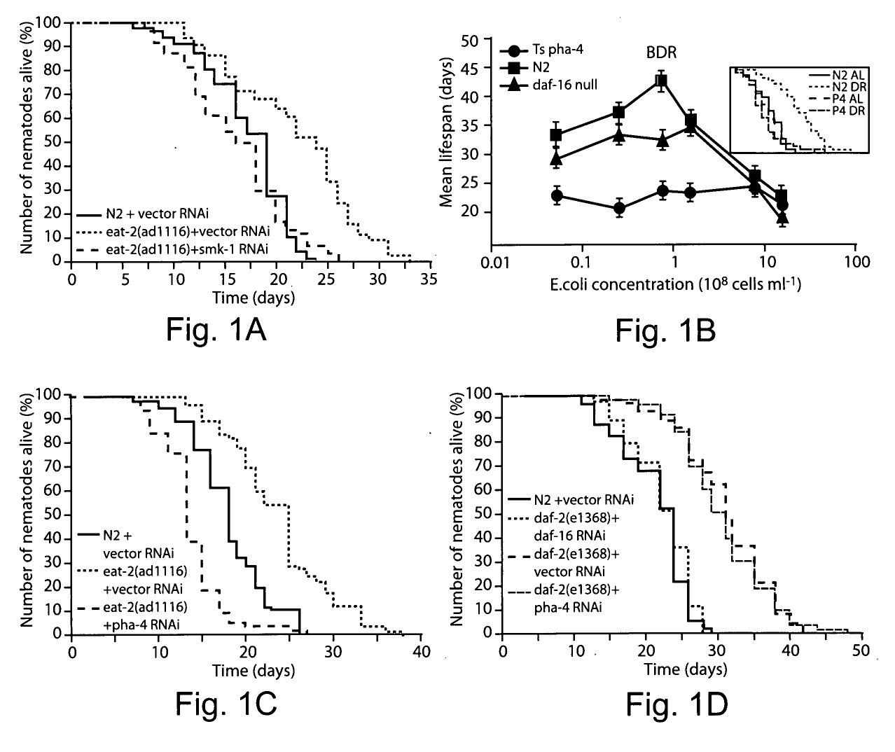

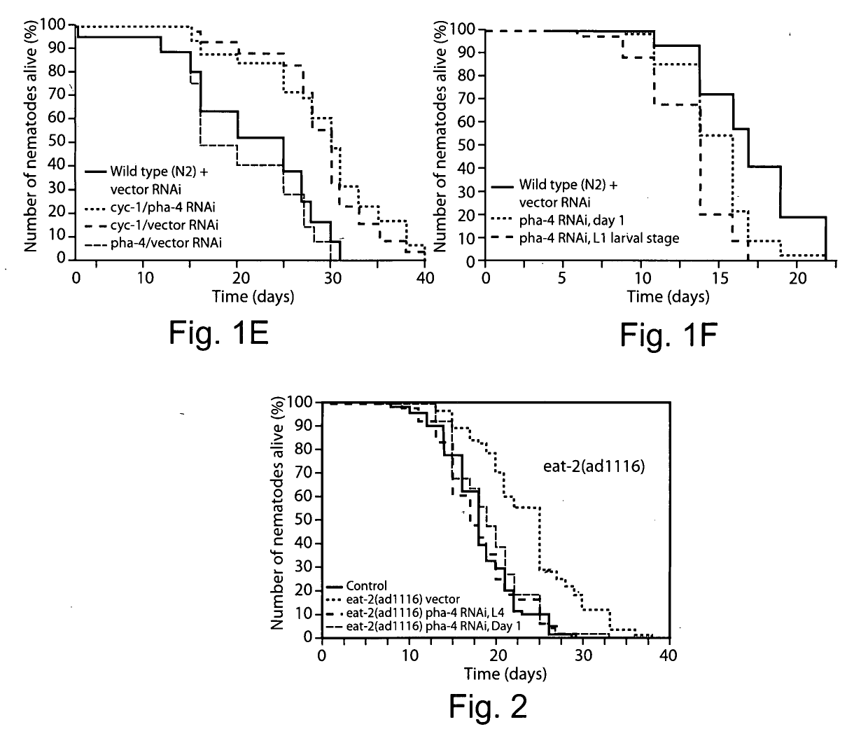

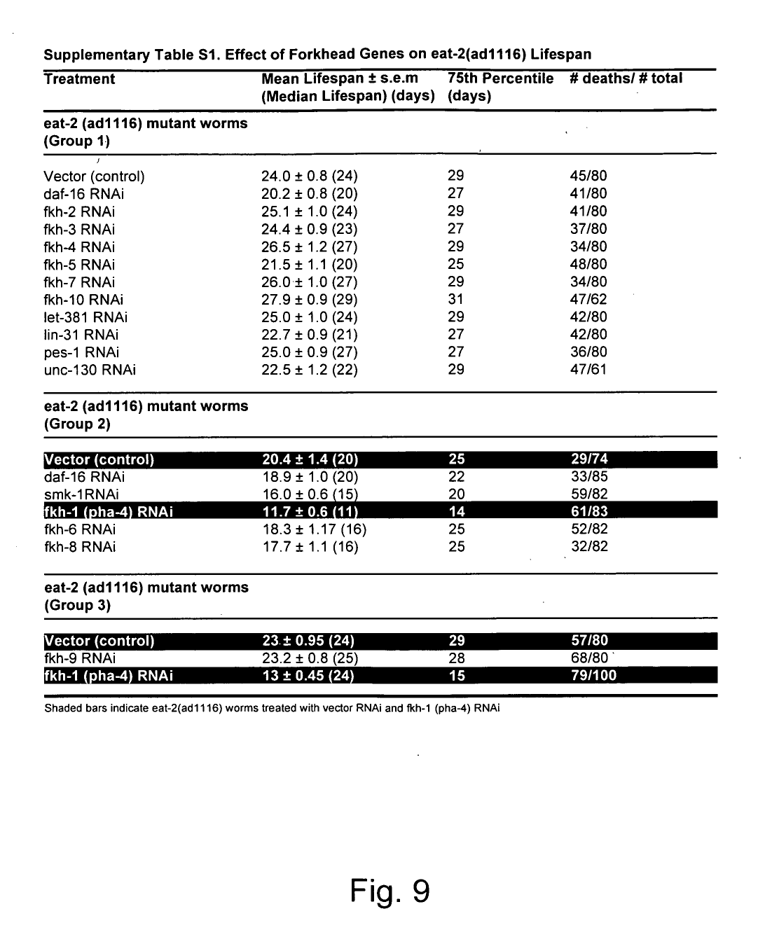

Each of fifteen forkhead-like genes found within the completed C. elegans genome (C. elegans sequencing consortium. Genome sequence of the nematode C. elegans: a platform for investigating biology. Science 282, 2012-2018 (1998)) were systematically inactivated to examine their role in dietary restriction. RNA interference (RNAi) of only one, pha-4, completely suppressed the long lifespan of eat-2(ad1116) mutant animals (FIG. 1C and the table in FIG. 9). The present invention provides methods of using pha-4 and its involvement in DR longevity to identify and use longevity modulators.

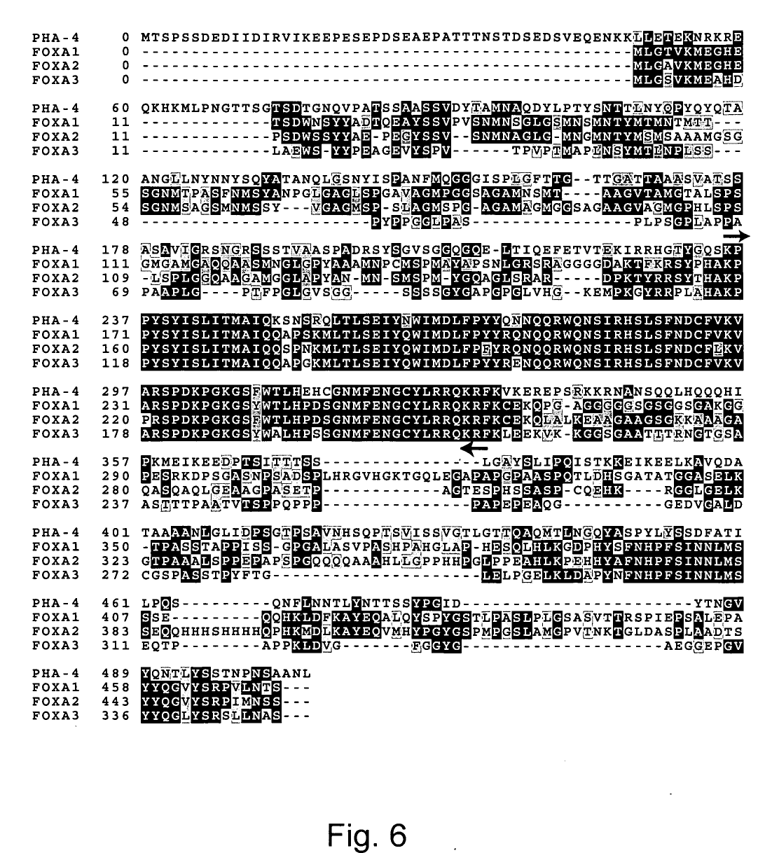

PHA-4 is orthologous to the human Foxa family of transcription factors (See, e.g., FIG. 6 and Horner, M. A. et al. pha-4, an HNF-3 homolog, specifies pharyngeal organ identity in Caenorhabditis elegans. Genes Dev. 12, 1947-1952 (1998)). Foxa1 homozygous mutant mice die shortly after birth, do not gain weight and are hypoglycaemic, suggesting an important role for Foxa1 in pancreatic cell function and a central role in metabolic homeostasis (Shih et al., Impaired glucose homeostasis and neonatal mortality in hepatocyte nuclear factor 3 deficient mice. Proc. Natl. Acad. Sci. USA 96, 10152-10157 (1999); and Kaestner et el. Inactivation of the winged helix transcription factor HNF3a affects glucose homeostasis and islet glucagon gene expression in vivo. Genes Dev. 13, 495-504 (1999)). Foxa2 is also required for glucagon expression in the pancreas and induction of gluconeogenic genes during fasting in the liver (Zhang et al. Foxa2 integrates the transcriptional response of the hepatocyte to fasting. Cell Metab. 2, 141-148 (2005)). Foxa3 mutant mice become hypoglycaemic after a prolonged fasting (See, Kaestner et al., Targeted disruption of the gene encoding hepatocyte nuclear factor 3c results in reduced transcription of hepatocyte-specific genes. Mol. Cell. Biol. 18, 4245-4251 (1998); Shen et al., Foxa3 (hepatocyte nuclear factor 3c) is required for the regulation of hepatic GLUT2 expression and the maintenance of glucose homeostasis during a prolonged fast. J. Biol. Chem. 276, 42812-42817 (2001)). The bifunctional role for Foxa family members in development and metabolic homeostasis of mammals prompted further investigation regarding a role for pha-4 in the regulation of metabolism and diet-restriction-mediated longevity of the adult worms in addition to its known role in development of the worm (See, Gaudet & Mango, Regulation of organogenesis by the Caenorhabditis elegans FoxA protein PHA-4. Science 295, 821-825 (2002); Mango et al., The pha-4 gene is required to generate the pharyngeal primordium of Caenorhabditis elegans. Development 120, 3019-3031 (1994)).

It will be appreciated that while the methods and compositions are predominantly discussed herein in terms of nematodes, they are also capable of use with other animals, including humans, e.g., other organisms that express a pha-4 ortholog or homolog, such as the foxa genes in mammals. Also, even though in certain embodiments the invention is directed towards particular configurations and/or combinations of such aspects, those of skill in the art will appreciate that not all embodiments necessarily comprise all aspects or particular configurations (unless specifically stated to do so).

### Definitions

Before describing the present invention in detail, it is to be understood that the invention herein is not necessarily limited to use with pha-4, but also includes orthologs and homologs of pha-4, e.g., foxa genes, as well as daf-16 genes and sod genes, and orthologs and homologs of those. It is also to be understood that the terminology used herein is for the purpose of describing particular embodiments only, and is not necessarily intended to be limiting. As used in this specification and the appended claims, the singular forms “a,” “an,” and “the” include plural referents unless the context clearly dictates otherwise. Thus, for example, reference to “a modulator” optionally includes a combination of two or more modulators, and the like.

Unless defined otherwise, all technical and scientific terms used herein have the same meaning as commonly understood by one of ordinary skill in the art to which the invention pertains. Although any methods and materials similar or equivalent to those described herein can be used in the practice of the present invention, the preferred materials and methods are described herein. In describing and claiming the present invention, the following terminology will be used in accordance with the definitions set out below.

“Longevity” and/or “lifespan” is used herein to refer to the length of subject's life, e.g., the number of years, days, minutes, etc., in the life span of an animal. The subjects of the invention are typically non-human animals, e.g., mice, nematodes, or flies, when screening methods are employed but are also optionally humans or other mammals when treatment or modulation of longevity is claimed. As used herein an “increase” or “modulation” of longevity also optionally includes a delay in the onset of age-related diseases and/or conditions and/or a delay and/or stabilization of the aging process.

“Dietary restriction” (DR) refers to restriction in caloric intake of an animal, e.g., a subject being tested for longevity modulation. Typically, an animal subjected to dietary restriction receives about 70% of its normal caloric intake, e.g., while receiving all necessary nutrients and vitamins. In some embodiments, an animal may receive only about 60% or about 50% of its normal caloric intake when subjected to dietary restriction. Dietary restriction, as used herein, also refers to models that are commonly used to simulate dietary restriction, such as mutant animals that are used as genetic surrogates of dietary restriction. The animals typically mimic dietary restriction with a reduced rate of pharyngeal pumping that is representative of reduced eating (see, e.g., Avery, L. The genetics of feeding in Caenorhabditis elegans. Genetics 133, 897-917 (1993); Lakowski & Hekimi. The genetics of caloric restriction in Caenorhabditis elegans. Proc. Natl. Acad. Sci. USA 95, 13091-13096 (1998)). Alternatively, in worms, dietary restriction takes the form of bacterial dietary restriction (BRD), which involves limiting the concentration of bacteria fed to worms in culture.

A “modulator” is a compound that modulates an activity of a given gene, protein, polypeptide, mRNA, or the like, e.g., to produce a phenotypic change such as increase in lifespan. The term “modulate” refers to a change in an activity or property of the gene or protein. For example, modulation can cause an increase or a decrease in one or more protein activity, and/or binding characteristic (e.g., binding of a transcription factor to a nucleic acid). The change in activity can arise from, for example, an increase or decrease in expression of one or more genes that encode these polypeptides, a change in stability of an mRNA that encodes the polypeptide, translation efficiency, or from a change in activity of the polypeptide itself. For example, a molecule that binds to a pha-4 gene or polypeptide can cause an increase or decrease in a biological activity of the polypeptide or expression of the gene. In addition, a modulator of the invention can result in a change in lifespan or a delay in the onset of an age-related diseases or condition. Example modulators include pha-4 and/or daf-16 agonists, antagonists, ligands, antibodies, or complexes thereof, etc. Modulators of the invention are identified, e.g., from a group of “test compounds” or “test agents” that include, but are not limited to, antibodies, proteins, nucleic acids, antisense molecules, small molecules, hormones, transcription factors, and the like.

The term “nucleic acid” encompasses any physical string of monomer units that can be correlated to a string of nucleotides, including a polymer of nucleotides (such as DNA or RNA), PNA, modified nucleotides, and the like. A nucleic acid is optionally double-stranded or single-stranded and any particular sequences referred to herein encompass the complementary sequence as well as the sequence which is explicity indicated. The terms “DNA” and “RNA” include, but are not limited to all single strand and double strand nucleic acid sequences or polynucleotides, such as cDNA, mRNA, antisense molecules, oligonucleotides, and the like. In addition, the nucleic acids of the invention include naturally occurring and non-naturally occurring nucleotide analogs and backbone substitutes, e.g., PNA, that one of skill in the art would recognize as capable of substituting for naturally occurring nucleotides and backbones of nucleic acids.

“Antisense” nucleic acids typically comprise DNA or RNA molecules that are complementary to at least a portion of an mRNA molecule. Antisense nucleic acids hybridize, e.g., in a cell, to a corresponding mRNA to form a double stranded molecule that interferes with translation of the mRNA. Antisense oligomers are typically at least about 15 to at least about 50 nucleotides. The use of antisense methods to inhibit the in vitro translation of genes is well known in the art (Marcus-Sakura Anal. Biochem. 172: 289, 1998). In the present case, animals transformed with constructs containing antisense fragments of the pha-4 and/or the daf-16 gene can display a modulated pha-4-related phenotype such as altered longevity.

The term “antibody” herein is used in the broadest sense and specifically covers monoclonal antibodies, polyclonal antibodies, multispecific antibodies (e.g. bispecific antibodies) formed from at least two intact antibodies, humanized antibodies, and antibody fragments so long as they exhibit a desired biological activity. An antibody is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. An intact antibody is one comprising heavy- and light-variable domains as well as an Fc region. Antibody fragments comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments. Antibody fragments are optionally produced using enzymatic digestion of intact antibodies or synthesized chemically or by recombinant DNA methods. The subunit structures and three-dimensional configurations of different classes and fragments of immunoglobulins are well known and the term antibody as used herein includes all configurations, fragments, and classes. Methods of making and using antibodies are well known to those of skill in the art.

A “pha-4 polypeptide” is a polypeptide that is the same as, a splice-variant of, or homologous to a naturally occurring pha-4 polypeptide, or that is derived from such a polypeptide (e.g., through cloning, recombination, mutation, or the like). The polypeptide can be full length or a fragment of a full length protein. A pha-4 fragment typically includes at least 10 contiguous amino acids corresponding to a native pha-4 protein, such as nematode pha-4 or a human foxa protein.

A “daf-16 polypeptide” is a polypeptide that is the same as, a splice-variant of, or homologous to a naturally occurring daf-16 polypeptide, or that is derived from such a polypeptide (e.g., through cloning, recombination, mutation, or the like). The polypeptide can be full length or can be a fragment of a full-length protein. A daf-16 fragment typically includes at least 10 contiguous amino acids corresponding to a native daf-16 protein, such as a daf-16 protein from C. elegans.

All polypeptides of the invention, e.g., pha-4, daf-16, sod proteins, foxa proteins, and the like can be naturally occurring or recombinant, and are optionally unpurified, purified, or isolated, and exist, e.g., in vitro, in vivo, or in situ.

A “pha-4 gene” or polynucleotide is a nucleic acid that encodes a pha-4 polypeptide. Typically, the gene includes regulatory sequences that direct expression of the gene in one or more cells of interest. Optionally, the gene is a native gene that includes regulatory and coding sequences that naturally direct expression of a pha-4 polypeptide, e.g., in a nematode or other animal. It is understood that polynucleotides encoding all or varying portions of pha-4 are included herein, as long as they encode a polypeptide with pha-4 activity, e.g., forkhead transcription factor activity. Such polynucleotides include naturally occurring, synthetic, and intentionally manipulated polynucleotides as well as splice variants. For example, portions of the mRNA sequence may be altered due to alternate RNA splicing patterns or the use of alternate promoters for RNA transcription.

A “daf-16” gene is a nucleic acid that encodes a daf-16 polypeptide. Typically, the gene includes regulatory sequences that direct expression of the gene in one or more cells of interest. Optionally, the gene is a native gene that includes regulatory and coding sequences that naturally direct expression of a daf-16 polypeptide, e.g., in a nematode such as C. elegans.

The term “sod” when used in relation to genes and/or polypeptides herein refers to a family of mitochondrial Fe/Mn superoxide dimutases, e.g., in C. elegans and any homologs or orthologs thereto.

The term “foxa” refers to a mammalian family of transcription factors, including foxa1, foxa2, and foxa3, that are orthologous to pha-4. The terms are used herein to include both the gene and its translated proteins. During development, the foxa gene is involved with specification of foregut endotherm in mammals and post development, in the upregulation of glucagon.

A cell or animal comprising pha-4 and/or daf-16 includes any sample comprising the gene, it's transcribed RNA, and/or translated polypeptides. As used herein, the terms, pha-4, daf-16, foxa, sod, and the like, refer to either the gene, the transcribed RNA, or its translated polypeptides, unless specifically stated otherwise. The term “pha-4” is also used herein to refer to a gene and its protein and any homologs or orthologs thereof. Therefore, whenever pha-4 is referred to, any of the mammalian foxa genes, e.g., foxa1, foxa2, or foxa3, is also contemplated. The same convention is used for all other genes and proteins discussed herein. Moreover, polynucleotides of the invention, e.g., pha-4, daf-16, or the like include polynucleotides having alterations in the nucleic acid sequence that still encode a polypeptide having the ability to modulate a pha-4 parameter such as longevity, lifespan and response to dietary restriction.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. For example, any naturally occurring pha-4 or other forkhead transcription factor can be modified by any available mutagenesis method to produce a mutant pha-4 transcription factor. Homology is generally inferred from sequence identity or similarity between two or more nucleic acids or proteins (or sequences thereof). The precise percentage of identity or similarity between sequences that is useful in establishing homology varies with the nucleic acid and protein at issue, but as little as 25% sequence similarity between proteins (and less between nucleic acids, due to the degeneracy of the genetic code) is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages (e.g., BLASTP and BLASTN using default parameters) are generally available. It is to be understood that the term “homolog,” as used herein includes orthologs of the sequence at issue as well.

“Orthologs” are genes in different species that evolved from a common ancestral gene by speciation. Normally, orthologs retain the same function in the course of evolution. As used herein “orthologs” are included in the term “homologs.”

The term “gene” is used broadly to refer to any nucleic acid associated with a biological function. Genes typically include coding sequences and/or the regulatory sequences required for expression of coding sequences. The term “gene” applies to a specific genomic sequence, as well as to a cDNA or an mRNA encoded by that genomic sequence. Genes also include non-expressed nucleic acid segments that, for example, form recognition sequences for other proteins. Non-expressed regulatory sequences include “promoters” and “enhancers,” to which regulatory proteins such as transcription factors bind, resulting in transcription of adjacent or nearby sequences. Genes of use in the present invention include, but are not limited to, pha-4, daf-16, foxa, sod, and other genes involved in longevity and dietary restriction pathways.

Expression of a gene or expression of a nucleic acid means transcription of DNA into RNA and optionally includes modification of the RNA, e.g., splicing, translation of RNA into a polypeptide (possibly including subsequent modification, e.g., posttranslational modification), and/or both transcription and translation. “Reduced expression,” e.g., of daf-16, refers to a situation in which a particular gene in a cell or animal is not translated at the same level as it would be in a wild-type or unmodified organism. Reduced expression includes a condition in which expression of the gene does not occur at all, e.g., a knock-out gene, and when expression levels are reduced as compared to the gene in a wild-type organism or cell. The “activity or expression level” of a gene is the level at which a gene is expressed in a cell or organism and can include the level at which the gene is transcribed and/or the level at which it is translated into a protein. Activity level is also used to refer to the activity of the protein, e.g., a transcription factor protein, and it's level of activity in a cell, e.g., enzymatic or binding activity of a protein encoded by the gene at issue.

A “pha-4 parameter” refers to a measurable parameter that is mediated by pha-4 such as longevity, lifespan and/or response to dietary restriction. In addition, a pha-4 parameter is optionally an activity or expression level of pha-4, either activity of a pha-4 polypeptide, transcription of a pha-4 gene into mRNA, or translation level or any gene expression or polypeptide that can be correlated to pha-4. For example, sod genes and daf-16 can be correlated to expression of pha-4 and daf-16 can be used as a pha-4 parameter because pha-4 longevity modulation is more pronounced in the absence of daf-16. Therefore, a change in daf-16 expression is also optionally used to determine whether a compound modulates longevity or pha-4 activity. A pha-4 parameter is also optionally an activity or expression level of a foxa gene, e.g., fox1, foxa2, foxa3, or any other homolog or ortholog of pha-4.

Aging is the accumulation of diverse adverse changes that increase the risk of death and/or disease, or quality of life. These changes can be attributed to development, genetic defects, the environment, disease, and the inborn aging process. The modulators or test agents of the present invention inhibit aging, e.g., in adult subjects, thereby increasing lifespan or longevity. In addition, the modulators of the present invention also protect against one or more age-related diseases in a subject. Such diseases include some types of cancer, such as prostate cancer and colon cancer, diabetes, neurodegenerative diseases, and the like. Delaying onset of these diseases or conditions, e.g., delaying the time at which a subject begins to exhibit symptoms of the diseases, can increase lifespan.

The term “age related disease” is used herein to refer to diseases, conditions and symptoms that are predominantly found or manifested in older animals, e.g., in humans, people over 50 or more preferably people over 65. For any animal, age-related diseases would manifest after maturation, e.g., post-development. The age at which maturity is reached is different depending on the animal and for each animal such time would be well known to those of skill in the art. Age-related diseases include certain cancers, atherosclerosis, diabetes (type 2), osteoporosis, hypertension, depression, Alzheimer's, Parkinson's, glaucoma, certain immune system defects, kidney failure, liver steatosis, and other conditions well known to those of skill in the art.

As used herein, the term “animal” refers to either a whole animal, an animal organ, an animal cell, or a group of animal cells, such as an animal tissue, for example, depending upon the context. Animals included in the invention are any animals amenable to transformation techniques, including vertebrate and non-vertebrate animals and mammals. Examples of mammals include, but are not limited to, pigs, cows, sheep, horses, cats, dogs, chickens, or turkeys. Other animals useful for screening methods include, but are not limited to, mice, rats, flies, and nematodes. When describing treatment and diagnostic procedures, the term “animal” in the present invention includes humans. For most screening procedures however, the animal used herein is a non-human animal, e.g., any animal other than a human.

A variety of additional terms are defined or otherwise characterized herein.

Dietary Restriction Induced Longevity is Mediated by pha-4 and daf-16

Dietary restriction is known to increase lifespan in a variety of animals, e.g., mice, flies, worms, and others. In the present invention, genes that mediate this process are described and screening methods for identifying modulators of those genes are presented. By identifying modulators of the genes that mediate this process, methods of modulating longevity and the compounds to do so are also provided.

Embodiments of the invention are based, in part, upon the identification of pha-4 as a mediator of dietary restriction induced longevity. By characterizing the role of pha-4 in dietary restriction and its relationship to daf-16 and other genes in the dietary restriction pathway, the present invention provides novel methods of identifying modulators of longevity, e.g., by identifying and using test compounds that modulate pha-4 to increase longevity.

In brief, the present invention shows that pha-4 is required for dietary restriction and not for other pathways and that the loss of pha-4 does not result in general sickness. In response to dietary restriction, expression of pha-4 is increased, resulting in increased longevity. Further, it is shown that the increase in longevity due to pha-4 is more pronounced in the absence of daf-16. Sod genes are also shown herein to be regulated by pha-4 and therefore are also optionally used to identify modulators of this longevity pathway. These findings are described in more detail below and in the example section.

Pha-4 is required for multiple forms of dietary restriction. RNA interference of the forkhead transcription factor gene pha-4 completely suppressed the long lifespan of eat-2(ad1116) worms, the mutant worms that are often used in various dietary restriction studies because they exhibit a reduced rate of pharyngeal pumping representative of eating. See, e.g., FIG. 1C. The mutants provide a genetic surrogate of dietary restriction. Because pha-4 is required for development of the worm pharynx, the role of pha-4 in dietary restriction was also tested in a non-genetic model, to determine whether pha-4 suppressed dietary restriction. In this model, dietary restriction is achieved by limiting the concentration of bacteria fed to non-mutant worms in culture (bacterial dietary restriction). In this model, the loss of pha-4 (which was delayed until adulthood to avoid possible development abnormalities) blocked the entire response to dietary restriction and the worms did not exhibit increased lifespan. See, e.g., FIG. 1B. Therefore, pha-4 is shown to be required for dietary restriction. Details of these experiments are provided in the examples below.

In addition to being required for dietary restriction induced longevity, pha-4 is also shown to be specific to diet-restriction-longevity. In other words, pha-4 is not required for other longevity pathways, e.g., the insulin/IGF-1 and mitochondrial electron transport longevity pathways. Furthermore, loss of pha-4 suppresses lifespan increases at many different bacterial concentrations and does not cause general sickness. In addition, it is shown that pha-4's role in longevity can be separated from its role in development. In the experiments described in more detail below, RNAi was used to inactivate pha-4 in adulthood. This allowed the worms being studied to develop normally. In all instances, loss of pha-4 was shown to suppress any longevity increase due to dietary restriction. Therefore, pha-4 is a useful target for screening for modulators of longevity.

Furthermore, expression of pha-4 is shown herein to be increased in response to dietary restriction. In addition, tests to determine whether overexpression of pha-4 was sufficient to increase lifespan under normal feeding conditions are provided. Surprisingly, it is shown that the greatest increase in longevity occurs when pha-4 is overexpressed in the absence of daf-16. Therefore, the present invention provides screening methods to identify compounds that both increase the expression of pha-4 and decrease the expression of daf-16. Such compounds are then optionally used to increase longevity, e.g., to treat a patient suffering from premature aging.

In an analysis of the relationship between daf-16 and pha-4, it is noted that the DNA binding sites for PHA-4 and DAF-16 overlap. In response to this overlap, target genes of daf-16 are analyzed to determine if any overlap exists in the genes regulated by pha-4 and daf-16. The mitochondrial Fe/Mn superoxide dimutases or “sod genes” of C. elegans are shown herein to be differentially regulated by daf-16 and pha-4. Sod-2 and sod-4 expression are specific to dietary restriction and dependent on pha-4. Sod-3 expression is specific to the insulin pathway and dependent on daf-16; and sod-1 and sod-5 are common to both pathways and regulated by both daf-16 and pha-4. This determination allows the sod genes and their expression levels to be used as indicators of pha-4 expression. For example, activity or expression levels of sod genes are optionally used as pha-4 parameters in the screening methods provided

All of the above analyses of pha-4 and daf-16 are described in more detail, including detailed methods, in the example section below. See also, Dillin et al. (2007) Nature, 447; 550-555 and online at doi:10.1038/nature05837.

### Screening for Longevity Modulators

In one aspect, methods of identifying compounds that modulate longevity and/or delay onset of age-related diseases or conditions, e.g., by modulating an activity or expression level of a pha-4 gene or polypeptide or other gene or protein involved in the dietary restriction pathway, are provided. In another aspect, the methods are used to identify compounds that increase longevity or delay onset of age-related diseases by modulation of both pha-4 and daf-16, e.g., an increase in pha-4 expression and a decrease in daf-16 expression together work to increase lifespan.

In these methods, a cell or non-human animal that expresses pha-4 or a homolog thereof is contacted with or administered a test compound and a pha-4 related parameter is assayed. Pha-4 parameters include, but are not limited to, expression of pha-4 or a homolog thereof, expression of daf-16 or a homolog thereof, expression of a sod gene or a homolog thereof, expression of a foxa gene or any homolog thereof, activity of pha-4 polypeptide, a sod polypeptide, a foxa polypeptide, or a daf-16 polypeptide, a change in lifespan, a delay in onset of an age related disease or condition, or binding of the test compound to one of the above genes or polypeptides or complexes thereof. By assaying changes in these parameters, compounds or combinations of compounds that modify, e.g., increase, longevity are identified. Compounds identified by these methods are also a feature of the invention.

Test compounds or agents for use in the methods provided include, but are not limited to, antibodies, proteins, nucleic acids, antisense molecules, small molecules, hormones, transcription factors, RNAi, ions, carbohydrates, organic or inorganic compounds, protein fragments, nucleic acid fragments, antibody fragments, and the like, and are optionally selected from natural or synthetic molecules. A test compound is optionally an antagonist of a dietary restriction pathway polypeptide or gene, an agonist of such polypeptides or genes, a ligand that specifically binds to a polypeptide or gene of the invention, an antibody that specifically binds to a polypeptide or gene in the pathway, or the like. For example, a test compound or modulator of the invention is optionally a ligand or other compound that increases expression of pha-4.

In general, test compounds that enhance activity or expression of pha-4 are desirable, e.g., to modulate lifespan, e.g., in response to dietary restriction. In addition, compounds that both increase activity or expression of pha-4 and decrease activity or expression of daf-16 are preferred for longevity modulation. Such compounds are optionally tested in the presence of dietary restrictions on the organism or without any dietary restrictions in place.

Identification of compounds that modulate, e.g., increase longevity or delay onset of age-related diseases, is optionally achieved by utilizing the genes and/or polypeptides of the invention, including active fragments thereof, in cell-based assays or whole organism assays, e.g., in nematodes. A variety of formats are applicable, including measurement of lifespan, or measurement of expression, e.g., using any of the pha-4 parameters described above.

### Cell Free Assays

In one embodiment, cell-free assays for identifying such compounds comprise a reaction mixture containing a pha-4 polypeptide or gene and optionally a daf-16 polypeptide or gene or homologs or orthologs thereof, and a test compound or a library of test compounds. In a cell-free assay, binding of the test compounds to the pha-4 polypeptides or genes is measured, e.g., to prescreen a library of compounds. Any compounds that specifically bind to pha-4 or daf-16 are then optionally tested in a cell based assay or a whole organism assay, e.g., for an effect on lifespan or gene expression. Detection of the formation of complexes is achieved by conventional methods well known to those of skill in the art.

For example, in one embodiment, a library of test compounds is synthesized on a solid substrate, e.g., a solid surface, plastic pins or some other surface. The test compounds are reacted with a polypeptide and/or gene and washed to elute unbound polypeptide. Bound polypeptide and/or gene is/are then detected by methods well known in the art. A reciprocal assay can also be used, e.g., in which the polypeptides and/or genes of interest, e.g., daf-16 and pha-4, are applied directly onto plates and binding of a test compound to the polypeptides or genes is detected. An antibody or other ligand binding to a polypeptide and/or gene of interest is optionally detected in either format. For example, a ligand that binds to a pha-4 gene or a pha-4 transcription factor polypeptide can be identified in this manner.

Interaction between molecules is also optionally assessed using real-time BIA (Biomolecular Interaction Analysis, e.g., using devices from Pharmacia Biosensor AB), which detect surface plasmon resonance (an optical phenomenon). Detection depends on changes in the mass concentration of macromolecules at the biospecific interface and does not require specific labeling of the molecules. In one useful embodiment, a library of test compounds is immobilized on a sensor surface, e.g., a wall of a micro-flow cell. A solution containing a pha-4 and/or daf-16 polypeptide or gene is then continuously circulated over the sensor surface. An alteration in the resonance angle, as indicated on a signal recording, indicates the occurrence of an interaction. This general technique is described in more detail in the BIAtechnology Handbook by Pharmacia.

Optionally, a pha-4 and/or daf-16 polypeptide or gene is immobilized to facilitate separation of complexes formed between the polypeptide or gene of interest and a test compound from uncomplexed forms of the polypeptide or gene. This also facilitates automation of the assay. Complexation of pha-4 and/or daf-16 can be achieved in any type of vessel, e.g., microtitre plates, microfluidic chambers or channels, micro-centrifuge tubes and test tubes. In one embodiment, a pha-4 or daf-16 polypeptide is fused to another protein, e.g., glutathione-S-transferase to form a fusion protein which is adsorbed onto a matrix, e.g., glutathione Sepharose™ beads (Sigma Chemical. St. Louis, Mo.), which are then combined with a test compound or test compound library and incubated under conditions sufficient to form test-compound-polypeptide complexes. Subsequently, the beads are washed to remove unbound label, and the matrix is immobilized and the radiolabel is determined. Similar methods for immobilizing proteins on matrices use biotin and streptavidin. For example, a protein can be biotinylated using biotin NHS (N-hydroxy-succinimide), using well known techniques and immobilized in the well of streptavidin-coated plates. The immobilized pha-4 or daf-16 is then used to test for binding of test compounds. Test compounds that are identified in cell free assays, e.g., by binding to a pha-4 or daf-16 polypeptide or gene, are then optionally screened further for modulation of longevity and/or expression of pha-4 and/or daf-16, e.g., in cell based or whole organism methods as described below.

### Cell Based Assays

In addition to cell-free assays such as those described above, cell-based assays are preferably used for identifying compounds that bind to, activate and/or modulate pha-4 and/or daf-16, and thereby increase longevity. The cell based assays of the invention typically comprise providing a cell that expresses pha-4 or a homolog thereof and contacting the cell with a test compound. The cells are then assayed for changes (e.g., as compared to a control cell without the test compound) in one or more pha-4 parameters, such as pha-4 expression, daf-16 expression, expression of sod genes, e.g., sod2 and/or sod4, or the like.

For example, a cell that expresses pha-4 and daf-16 or homologs thereof is optionally used to assay for compounds that increase expression of pha-4 and decrease expression of daf-16. Alternatively, a cell that expresses pha-4 and exhibits reduced expression if daf-16 is used to identify modulators of pha-4, e.g., modulators that increase expression of pha-4. The cells are exposed to or contacted with a test compound or library of test compounds and a pha-4 parameter is measured or assayed. For example, formation of a complex between the test compound and pha-4 or daf-16 is optionally assayed or expression of pha-4 is measured by measuring amount of transcribed mRNA.

Cells that are useful for the screening methods of the invention can be mammalian cells, yeast cells, bacterial cells, insect cells, Xenopus oocytes, human or other mammalian cells, or any other cell expressing pha-4 and daf-16 or homologs thereto, whether that expression is natural to the cell or, more typically, the result of recombinant introduction of a pha-4 and/or daf-16 gene of interest into the cell. Further details regarding appropriate cells, sources of genes of interest, etc., are provided below.

Expression of pha-4 or any other pha-related gene, such as daf-16, can be detected, e.g., via northern analysis or quantitative (e.g., real time) RT-PCR, before and after application of potential expression modulators. Similarly, promoter regions of pha-4 and/or daf-16 gene(s) of interest (e.g., generally sequences in the region of the start site of transcription, e.g., within 5 KB of the start site, e.g., 1 KB, or less e.g., within 500 BP or 250 BP or 100 BP of the start site) can be coupled to reporter constructs (CAT, beta-galactosidase, luciferase or any other available reporter) and can be similarly tested for expression activity modulation by the potential modulator. In either case, the assays can be performed in a high-throughput fashion, e.g., using automated fluid handling and/or detection systems, in a serial or parallel fashion.

For example, the level of expression of pha-4 and/or daf-16, sod, or the like, is optionally measured by measuring the amount of translated protein, which is optionally measured using immunoassays such as western blotting, ELISA, and the like. Pha-4 proteins are optionally analyzed by standard SDS-PAGE and/or immunoprecipitation analysis.

Another way to assess the level of expression of a pha-4 parameter gene is by measurement of pha-4 polynucleotides, e.g., transcribed mRNA, e.g., using amplication, such as PCR or LCR, or hybridization assays. Polynucleotide sequences of the invention include DNA, cDNA and RNA sequences which encode pha-4 and/or daf-16 or homologs thereof.

In any of the assays herein, control compounds are optionally administered and the activity of the control compounds compared to those of the test compounds to verify that changes in activity resulting from application of the test compound are not artifacts. For example, control compounds can include various dyes, buffers, adjuvants, carriers, or the like that the test compounds are typically administered with, but lack a putative test compound.

Test compounds identified in cell-based assays as modulators of a pha-4 parameter, e.g., pha-4 expression or sod2 expression, are then optionally further assayed for lifespan modulation in a whole organism assay as described below.

### Whole Organism Assays

In another embodiment, the present invention provides screening assays in whole organisms. In a whole organism screening assay, as in the cell based systems described above, modulators for longevity are identified. The methods typically comprise providing an organism for screening, administering a test compound to the organism, and assaying for a change in one or more pha-4 parameter due to the presence of the test compound.

The organisms for use in the assays are typically non-human animals, including both vertebrates and invertebrates, mammals and other animals. Typically, the animals are adult, e.g., mature, post-development, animals. Preferred organisms for screening for longevity modulators are flies (e.g., Drosophila) and worms, e.g., C. elegans, as well as typical mammalian laboratory animals such as mice, rabbits, and rats. The organisms are optionally wild-type organisms or transgenic animals as described herein. Typically, the organisms express pha-4 or a homolog or ortholog thereof, e.g., a foxa ortholog. The organisms also optionally express daf-16, e.g., at a reduced or knock-out level, or a wild-type level.

The organisms are then administered a test compound, e.g., a library of test compounds is administered to an array of organisms. Administration of the test compound is optionally by injection or by feeding the compound to the animal, or any other mode of administration that is optionally used for pharmaceuticals. The animals are also optionally subjected to dietary restriction, e.g., reduced caloric intake, during the assay.

To assay a pha-4 parameter, the organisms are optionally observed to determine lifespan, e.g., a mean or median lifespan. Alternatively, expression levels of any of a variety of genes is measured. For example, expression of both daf-16 and pha-4 is optionally measured, with an increase in pha-4 expression and a decrease in daf-16 expression indicative of a longevity increase. Alternatively, assays to detect increases in expression of pha-4 are carried out in organisms that exhibit reduced expression of daf-16 or that do not express daf-16 at all. Any pha-4 parameter as described herein is optionally monitored by any method known to those of skill in the art with changes in the parameters indicative of longevity modulation. Other genes that are useful for pha-4 parameters include, but are not limited to, sod genes, foxa genes, and the like.

In a preferred embodiment, the roundworm Caenorhabditis elegans is used to assay for longevity modulators. C. elegans is a simple soil nematode species that has been extensively described at the cellular and molecular level, and is a model organism for biological studies. C. elegans can develop through a normal life cycle that involves four larval stages and a final molt into an adult hermaphrodite. The dauer pathway is an alternative life cycle stage common to many nematode species which is normally triggered by environmental stresses such as starvation, temperature extremes, or overcrowding. Genetically, the dauer pathway has been most intensively studied in C. elegans. However, in the present invention, the dauer pathway is typically avoided and adult nematodes are used, to avoid any interference between development pathways and longevity pathways.

In one embodiment, the pha-4 parameter comprises increased or decreased life span. Life span assays have been well described in the art. (See, e.g., Apfeld J. & Kenyon C. (1998) Cell 95: 199-210). In organisms that exhibit a modified life span, an agent is identified based on its ability to either further extend or shorten the lifespan. Resistance to ultraviolet (UV) stress is determined, e.g., by exposing the organism to UV light and measuring life span from the day of UV treatment. Oxidative stress resistance is determined by exposing the animals to paraquat, which produces superoxide when taken up by cells, and determining survival from the day of treatment (See, e.g., Feng et al. (2001) Dev. Cell 1:1-20.). Heat tolerance is measured by exposing adult animals to a 35° C. heat shock for 24 hours, and then assaying the animals for viability. Alternatively, animals that express a reporter gene are used, wherein the reporter gene can be detected in living animals (e.g., GFP), e.g., with a machine that could monitor the animals using a suitable reporter gene detection protocol.

After identification of a modulator, e.g., by measuring expression levels of pha-4 and daf-16, additional assays are optionally conducted using the compound identified to further characterize the nature of the modulators action with respect to longevity. Further studies of lifespan are optionally conducted and, egg laying can be measured to determine whether the longevity occurs by delaying maturity. Compounds identified using these methods are then optionally used as the active ingredient in pharmaceuticals that extend lifespan, e.g., to treat premature aging or delay onset of age related diseases.

### High Throughput Screening and Screening Systems

High throughput methods of screening, e.g., drug screening, are particularly useful in identifying longevity modulators, e.g., modulators of pha-4 or daf-16 polypeptide activity or gene expression. Generally in these methods, one or more sample, e.g., a cell or animal that expresses pha-4 or a homolog thereof and daf-16 or a homolog thereof, is contacted with a plurality of test compounds. Modulation of the polypeptide or gene or of the lifespan of the organism by the test compounds is detected, thereby identifying one or more compound that binds to or modulates activity of the polypeptide, complex and/or gene. The assay methods of the present invention can be useful in performing high-throughput (greater than 1,000 compounds/day) and even ultra-high throughput (e.g., greater than 10,000 compounds/day) screening of chemical libraries, e.g., searching for modulators of longevity. These experiments may be carried out in parallel by a providing a large number of reaction mixtures (e.g., cell suspensions or organisms) in separate receptacles, typically in a multiwell format, e.g., 96 well, 324 well or 1536 well plates. Different test compounds (library members) are added to separate wells, and the effect of the compound on the reaction mixture is ascertained, e.g., via expression of a pha-4 gene of interest or lifespan of the organism. These parallelized assays are generally carried out using specialized equipment to enable simultaneous processing of large numbers of samples, i.e., fluid handling by robotic pipettor systems and detection in multiplexed systems

Essentially any available compound library can be screened in such a high-throughput format against a cell or organism expressing pha-4 and optionally daf-16, e.g., at reduced or wild-type levels. The effect of the library members on the activity level of the polypeptides or expression level of the genes is assessed, optionally in a high-throughput fashion, e.g., and compared to a control sample to which no test compound has been administered. Many libraries of compounds are commercially available, e.g., from the Sigma Chemical Company (Saint Louis, Mo.), Aldrich chemical company (St. Louis Mo.), and many can be custom synthesized by a wide range of biotech and chemical companies.

Automated systems of the invention can facilitate the screening methods described above (both in vitro and in vivo screening methods). That is, systems that facilitate cell or whole organism based screening for pha-4 and/or daf-16 expression and/or activity, lifespan, or delay of onset of age-related diseases are a feature of the invention. Similarly, systems designed to monitor physiological responses of animals, including non-human transgenic laboratory animals, are also a feature of the invention. System features herein are generally applicable to the methods herein and vice-versa.

High-throughput automated systems that detect compounds that bind to and/or modulate activity of a pha-4 and daf-16 typically include an array of samples, e.g., any cell or animal described herein or known to those of skill in the art that expresses or can be modified to express pha-4 and/or daf-16 or any homologs thereof. For example, nematodes that express pha-4 and daf-16, or that have been modified to express pha-4 but not daf-16, are optionally used to screen for compounds, e.g., drug candidates, that modulate longevity. A source of a plurality of test compounds is also typically included in such a system. A detector or monitoring module detects any changes in a pha-4 parameter in the samples, e.g., after contact with a test compound, and a correlation module correlates any changes that occur with longevity modulation and the particular compound that initiated such change.

For example, an array of nematodes in an array of containers is optionally provided, wherein the nematodes express pha-4 or a homolog thereof. The nematodes also optionally express daf-16 or a homolog thereof either at a normal or reduced level. Alternatively the nematodes do not express daf-16 at all. After the nematodes are exposed to, e.g., injected with or fed, a test compound, a monitoring module is used to detect changes in a pha-4 parameter. For example, modulation in a level or activity of a polypeptide or mRNA transcript(s) corresponding to pha-4 or a pha-4 parameter by the test compound is measured, thereby identifying a putative modulator of longevity. Alternatively, the lifespan of the nematodes is optionally measured by the monitoring module. After monitoring the pha-4 parameter, a correlation module, e.g., a computer, is then used to correlate changes in any of the pha-4 parameters to lifespan increases or decreases, thereby identifying any test compounds that modulate longevity

The source of test compounds for such systems and methods of the invention can be any commercially available or proprietary library of materials, including compound libraries from Sigma (St. Louis Mo.), Aldrich (St. Louis Mo.), Agilent Technologies (Palo Alto, Calif.) or the like. Those of skill in the art will be familiar with various sources and libraries of compounds suitable for drug screening.

The format of the library will vary depending on the system to be used. In one typical embodiment, libraries of sample materials are arrayed in microwell plates (e.g., 96, 384 or more well plates), which can be accessed by standard fluid handling robotics, e.g., using a pipettor or other fluid handler with a standard ORCA robot (Optimized Robot for Chemical Analysis) available from Beckman Coulter (Fullerton, Calif.). Standard commercially available workstations such as the Caliper Life Sciences (Hopkinton, Mass.) Sciclone ALH 3000 workstation and Rapidplate™ 96/384 workstation provide precise 96 and 384-well fluid transfers in a small, highly scalable format. Plate management systems such as the Caliper Life Sciences Twister® II Advanced Capability Microplate Handler for End-Users, OEM's and Integrators provide plate handling, storage and management capabilities for fluid handling, while the Presto™ AutoStack provides fast reliable access to consumables presenting trays of tips, reagents, microplates or deep wells to an automated device (e.g., the ALH 3000) without robotic arm intervention.

Microfluidic systems for handling and analyzing microscale fluid samples, e.g., in cell based and non-cell based approaches that can be used for analysis of test compounds on biological samples in the present invention are also available, e.g., the Caliper Life Sciences various LabChip® technologies (e.g., LabChip® 90 and 3000) and Agilent Technologies (Palo Alto, Calif.) 2100 and 5100 devices. Similarly, interface devices between microfluidic and standard plate handling technologies are also commercially available. For example, the Caliper Technologies LabChip® 3000 uses “sipper chips” as a “chip-to-world” interface that allows automated sampling from microtiter plates. To meet the needs of high-throughput environments, the LabChip® 3000 employs four or even twelve sippers on a single chip so that samples can be processed, in parallel, up to twelve at a time. Solid phase libraries of materials can also be conveniently accessed using sipper or pipetting technology, e.g., solid phase libraries can be gridded on a surface and dried for later rehydration with a sipper or pipette and accessed through the sipper or pipette. These sources are optionally used with whole organism, cell based, and/or cell free screening systems. For example, a library of test compounds in a microtiter plate can be accessed via a sipper or pipettor device into an array of containers or microchannels containing cells or organisms for screening as described above for pha-4 modulation.

As already noted, with regard to the systems and methods of the invention, the particular libraries of compounds can be any of those that now exist, e.g., those that are commercially available, or that are proprietary. A number of libraries of test compounds exist, e.g., those from Sigma (St. Louis Mo.), and Aldrich (St. Louis Mo.). Other current compound library providers include Actimol (Newark Del.), providing e.g., the Actiprobe 10 and Actiprobe 25 libraries of 10,000 and 25,000 compounds, respectively; BioMol (Philedelphia, Pa.), providing a variety of libraries, including natural compound libraries and the Screen-Well™ Ion Channel ligand library which are usefully screened for longevity modulators as described herein, as well as several other application specific libraries; Enamine (Kiev, Ukranie) which produces custom libraries of billions of compounds from thousands of different building blocks, TimTec (Newark Deleware), which produces general screening stock compound libraries containing >100,000 compounds, as well as template-based libraries with common heterocyclic lattices, libraries for targeted mechanism based selections, including kinase modulators, GPCR Ligands, channel modulators, etc., privileged structure libraries that include compounds containing chemical motifs that are more frequently associated with higher biological activity than other structures, diversity libraries that include compounds pre-selected from available stocks of compounds with maximum chemical diversity, plant extract libraries, natural products and natural product-derived libraries, etc; AnalytiCon Discovery (Germany) including NatDiverse (natural product analogue screening compounds) and MEGAbolite (natural product screening compounds); Chembridge (San Diego, Calif.) including a wide array of targeted or general and custom or stock libraries; ChemDiv (San Diego, Calif.) providing a variety of compound diversity libraries including CombiLab and the International Diversity Collection; Comgenix (Hungary) including ActiVerse™ libraries; MicroSource (Gaylordsville, Conn.) including natural libraries, agro libraries, the NINDS custom library, the genesis plus library and others; Polyphor (Switzerland) including privileged core structures as well as novel scaffolds; Prestwick Chemical (Washington D.C.), including the Prestwick chemical collection and others that are pre-screened for biotolerance; Tripos (St. Louis, Mo.), including large lead screening libraries; and many others. Academic institutions such as the Zelinsky Institute of Organic Chemistry (Russian Federation) also provide libraries of considerable structural diversity that can be screened in the methods of the invention, e.g., to find longevity modulators.

### System Components

Although the devices and systems specifically illustrated herein are generally described in terms of the performance of a few or one particular operation, it will readily be appreciated from this disclosure that these systems permit easy integration of additional operations. For example, the systems described will optionally include structures, reagents and systems for performing virtually any number of operations both upstream and downstream from the operations specifically described herein. Such upstream operations include sample handling and preparation operations, e.g., cell separation, extraction, purification, culture, amplification, cellular activation, labeling reactions, dilution, aliquotting, and the like. Downstream operations may also include similar operations, including, e.g., separation of sample components, labeling of components, assays and detection operations, movement of components into contact with cells, or organisms, or the like.

Upstream and downstream assay and detection operations include, without limitation, cell fluorescence assays, cell activity assays, receptor/ligand assays, immunoassays, lifespan determination, and the like. Any of these elements can be incorporated into the systems herein.

In general in the present invention, materials such as cells and organisms are optionally monitored and/or detected so that lifespan can be determined. Depending on the measurement made, decisions can be made regarding subsequent operations, e.g., whether to further assay a particular modulator in detail to determine the extent of life-span modulation, such as whether diet restriction is necessary for the modulator to work and whether the modulator also delays onset of age-related diseases.

The systems described herein generally include fluid handling devices, as described above, in conjunction with additional instrumentation for controlling fluid transport, flow rate and direction within the devices, detection instrumentation for detecting or sensing results of the operations performed by the system, processors, e.g., computers, for instructing the controlling instrumentation in accordance with preprogrammed instructions, receiving data from the detection instrumentation, and for analyzing, storing and interpreting the data, and providing the data and interpretations in a readily accessible reporting format.

Controllers

A variety of controlling instrumentation is optionally utilized in conjunction with the fluid handling elements described above, for controlling the transport and direction of fluids and/or materials (samples, cells, test compounds, etc.) within the systems of the present invention. Controllers typically include appropriate software to direct transport of organisms, fluid material, etc. in response to user instructions. For example, software that directs the amount of nutrition/food to be fed to an array of nematodes can be included in a system of the present invention to allow a user to alter the dietary restriction profile for an array of nematodes being screened. Software that allows a user to direct a particular RNAi molecule to be administered to a cell or organism allows a user to selectively knock-out a gene, such as pha-4 and/or daf-16 when screening for longevity modulators.

Typically, the controller systems are appropriately configured to receive or interface with a fluid handling or other system element as described herein. For example, the controller and/or detector, optionally includes a stage upon which a sample is mounted to facilitate appropriate interfacing between the controller and/or detector and the rest of the system. Typically, the stage includes an appropriate mounting/alignment structural elements, such as a nesting well, alignment pins and/or holes, asymmetric edge structures (e.g., to facilitate proper alignment of slides, microwell plates or microfluidic “chips”), and the like.

Detectors

Within the systems of the invention, detectors can take any of a variety of forms. The various fluid handling stations noted above often come with integrated detectors, e.g., optical or fluorescent detectors. However, other detectors such as one that measures lifespan is also optionally used.

System signal detectors are typically disposed adjacent to a site of reaction or mixing between a cell sample or organism and a test compound. This site can be a test tube, microwell plate, microfluidic device, or the like. The site is within sensory communication of the detector. The phrase “within sensory communication” generally refers to the relative location of the detector that is positioned relative to the site so as to be able to receive a particular relevant signal from that container. In the case of optical detectors, e.g., fluorescence, FRET, or fluorescence polarization detectors, sensory communication typically means that the detector is disposed sufficiently proximal to the container that optical, e.g., fluorescent signals, are transmitted to the detector for adequate detection of those signals. Typically this employs a lens, optical train or other detection element, e.g., a CCD, that is focused upon a relevant portion of the container to efficiently gather and record these optical signals.

Example detectors include patch-clamp stations, photo multiplier tubes, spectrophotometers, a CCD array, a scanning detector, a microscope, a galvo-scann or the like. Cells, dyes or other components which emit a detectable signal can be flowed past or moved into contact with the detector, or, alternatively, the detector can move relative to an array of samples (or, the detector can simultaneously monitor a number of spatial positions corresponding to samples, e.g., as in a CCD array). For example, a microscope is optionally moved relative to an array of nematodes in containers, e.g., to determine whether the organism in a particular container is alive at any given time point.

The system typically includes a signal detector located proximal to the site of mixing/reaction. The signal detector detects the detectable signal, e.g., for a selected length of time (t). For example, the detector can include a spectrophotometer, or an optical detection element. Commonly, the signal detector is operably coupled to a computer, which deconvolves the detectable signal to provide an indication of a pha-4 parameter, such as lifespan. Changes in the expression levels or activity of any pha-4 polypeptides or genes are monitored in response to a test compound (e.g., putative modulator), e.g., as compared to a control that does not include the test compound.

Computer

In screening systems of the invention, either or both of the controller system and/or the detection system are optionally coupled to an appropriately programmed processor or computer which functions to instruct the operation of these instruments in accordance with preprogrammed or user input instructions, receive data and information from these instruments, and interpret, manipulate and report this information to the user. As such, the computer is typically appropriately coupled to one or both of these instruments (e.g., including an analog to digital or digital to analog converter as needed).

The computer typically includes appropriate software for receiving user instructions, either in the form of user input into a set parameter fields, or in the form of preprogrammed instructions, e.g., preprogrammed for a variety of different specific operations. For example, a user may input the amount of nutrients a nematode is to receive or the pha-4 parameter that is to be measured. The software then converts these instructions to appropriate language for instructing, e.g., the operation of the fluid direction and transport controller to carry out further desired operations. The computer then receives the data from the one or more sensors/detectors included within the system, and interprets the data, either provides it in a user understood format, or uses that data to initiate further controller instructions, in accordance with the programming, e.g., such as in monitoring and control of flow rates, temperatures, further assays, and the like. For example, the computer correlates which test compounds are capable of binding to pha-4 in a cell free assay and then selects and initiates instruction that those compounds be used in cell-based assays that measure expression levels or whole organism assays that measure lifespan.

Biosensors

Biosensors of the invention are devices or systems that comprise the polypeptides or nucleic acids of the invention (e.g., a pha-4 or daf-16 polypeptide or nucleic acid) coupled to a readout that measures or displays one or more activity of the polypeptide or nucleic acid. Thus, any of the above described assay components can be configured as a biosensor by operably coupling the appropriate assay components to a readout. The readout can be optical (e.g., to detect cell markers, ion-sensitive dyes, cell potential, or cell survival) electrical (e.g., coupled to a FET, a BIAcore, or any of a variety of others), spectrographic, or the like, and can optionally include a user-viewable display (e.g., a CRT or optical viewing station). The biosensor can be coupled to robotics or other automation, e.g., microfluidic systems, that direct contact of the test compounds to the proteins of the invention, e.g., for automated high-throughput analysis of test compound activity. A large variety of automated systems that can be adapted to use with the biosensors of the invention are commercially available. For example, automated systems have been made to assess a variety of biological phenomena, including, e.g., expression levels of genes in response to selected stimuli (Service (1998) “Microchips Arrays Put DNA on the Spot” Science 282:396-399). Laboratory systems can also perform, e.g., repetitive fluid handling operations (e.g., pipetting) for transferring material to or from reagent storage systems that comprise arrays, such as microtiter trays or other chip trays, which are used as basic container elements for a variety of automated laboratory methods. Similarly, the systems manipulate, e.g., microtiter trays, and control a variety of environmental conditions such as temperature, exposure to light or air, and the like. Many such automated systems are commercially available. Examples of automated systems are available from Caliper Technologies (including the former Zymark Corporation, Hopkinton, Mass.), which utilize various Zymate systems that typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). A number of automated approaches to high-throughput activity screening are provided by the Genomics Institute of the Novartis Foundation (La Jolla, Calif.); See GNF.org on the world-wide web. Microfluidic screening applications are also commercially available from Caliper Technologies Corp. For example, (e.g., LabMicrofluidic Device® high throughput screening system (HTS) by Caliper Technologies, Mountain View, Calif. or the HP/Agilent technologies Bioanalyzer using LabChip™ technology by Caliper Technologies Corp. can be adapted for use in the present invention.

In an alternate embodiment, conformational changes are detected by coupling the polypeptides or complexes of the invention to an electrical readout, e.g., to a chemically coupled field effect transistor (a CHEM-FET) or other appropriate system for detecting changes in conductance or other electrical properties brought about by a conformational shift by the protein of the invention, e.g., by binding of a test compound to a polypeptide or gene of the invention.

Further Details Regarding Cells Expressing pha-4 and/or daf-16

Cells to be tested for changes in pha-4 and/or daf-16 expression or concentration are optionally derived from cell preparations. The cells can be those associated with pha-4/daf-16 expression in vivo, such as intestinal or neuronal cells. Alternately, the cells can be derived from such cells, e.g., through culture.

However, one feature of the invention is the production of recombinant cells, e.g., expressing a heterologous pha-4 gene, or both a heterologous pha-4 gene and a heterologous daf-16 gene. In these embodiments, the biological sample to be tested is derived from the recombinant cell, which is selected largely for ease of culture and manipulation. The cells can be, e.g., human, rodent, insect, Xenopus, etc. and will typically be a cell in culture (or an oocyte in the case of Xenopus).

pha-4 and daf-16 nucleic acids are typically introduced into cells in cloning and/or expression vectors to facilitate introduction of the nucleic acid and expression of pha-4 and/or daf-16 to produce pha-4 and/or daf-16 polypeptides. Vectors include, e.g., plasmids, cosmids, viruses, YACs, bacteria, poly-lysine, etc. A “vector nucleic acid” is a nucleic acid molecule into which a heterologous nucleic acid is optionally inserted which can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) artificial chromosomes. “Expression vectors” are vectors that comprise elements that provide for or facilitate transcription of nucleic acids which are cloned into the vectors. Such elements can include, e.g., promoters and/or enhancers operably coupled to a nucleic acid of interest.

In general, appropriate expression vectors are known in the art. For example, pET-14b, pcDNA1Amp, and pVL1392 are available from Novagen and Invitrogen and are suitable vectors for expression in E. coli, COS cells and baculovirus infected insect cells, respectively. pcDNA-3, pEAK, and vectors that permit the generation of pha-4 and/or daf-16 RNA for in vitro and in vivo expression experiments (e.g., in vitro translations and Xenopus oocyte injections) are also useful. These vectors are illustrative of those that are known in the art. Suitable host cells can be any cell capable of growth in a suitable media and allowing purification of the expressed protein. Examples of suitable host cells include bacterial cells, such as E. coli, Streptococci, Staphylococci, Streptomyces and Bacillus subtilis cells; fungal cells such as yeast cells, e.g., Pichia, and Aspergillus cells; insect cells such as Drosophila S2 and Spodoptera Sf9 cells, mammalian cells such as CHO, COS, and HeLa; and even plant cells.

Cells are transformed with pha-4 and/or daf-16 genes according to standard cloning and transformation methods. Such genes can also be isolated from resulting recombinant cells using standard methods. General texts which describe molecular biological techniques for making nucleic acids, including the use of vectors, promoters and many other relevant topics, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3nd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)).

In addition, a plethora of kits are commercially available for the preparation, purification and cloning of plasmids or other relevant nucleic acids from cells, (see, e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms, or the like.

As noted, typical vectors contain transcription and translation terminators, transcription and translation initiation sequences, and promoters useful for regulation of the expression of the particular target nucleic acid, e.g., pha-4 or daf-16. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both. See, Giliman & Smith, Gene 8:81 (1979); Roberts, et al., Nature, 328:731 (1987); Schneider, B., et al., Protein Expr. Purif. 6435:10 (1995); Ausubel, Sambrook, Berger (above). A catalogue of Bacteria and Bacteriophages useful for cloning is provided, e.g., by the ATCC, e.g., The ATCC Catalogue of Bacteria and Bacteriophage published yearly by the ATCC. Additional basic procedures for sequencing, cloning and other aspects of molecular biology and underlying theoretical considerations are also found in Watson et al. (1992) Recombinant DNA Second Edition, Scientific American Books, N.Y.

In addition, essentially any nucleic acid (and virtually any labeled nucleic acid, whether standard or non-standard) can be custom or standard ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others.

Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N.Y.); and Atlas and Parks (eds) The Handbook of Microbiological Media (1993) CRC Press, Boca Raton, Fla.

### Regulating Expression of Dietary Restriction Related Genes

Expression (e.g., transcription and/or translation) of daf-16 or pha-4 can be regulated using any of a variety of techniques known in the art. For example, gene expression can be inhibited using an antisense nucleic acid or an interfering RNA (RNAi), e.g., to decrease or knock out expression of daf-16. Inhibition of expression in particular cell-types can be used for further studying the in vitro or in vivo role of these genes, and/or as a mechanism for treating a condition caused by overexpression of a pha-4 or daf-16 gene, and/or for treating a dominant effect caused by a particular allele of such a gene. For example, expression of daf-16 is optionally reduced or silenced in cells used for screening for pha-4 related longevity modulators in the present invention.

For example, use of antisense nucleic acids is well known in the art. An antisense nucleic acid has a region of complementarity to a target nucleic acid, e.g., a target gene, mRNA, or cDNA. Typically, a nucleic acid comprising a nucleotide sequence in a complementary, antisense orientation with respect to a coding (sense) sequence of an endogenous gene is introduced into a cell. The antisense nucleic acid can be RNA, DNA, a PNA or any other appropriate molecule. A duplex forms between the antisense sequence and its complementary sense sequence, resulting in inactivation of the gene. The antisense nucleic acid can inhibit gene expression by forming a duplex with an RNA transcribed from the gene, by forming a triplex with duplex DNA, etc. An antisense nucleic acid can be produced, e.g., for any gene whose coding sequence is known or can be determined by a number of well-established techniques (e.g., chemical synthesis of an antisense RNA or oligonucleotide (optionally including modified nucleotides and/or linkages that increase resistance to degradation or improve cellular uptake) or in vitro transcription). Antisense nucleic acids and their use are described, e.g., in U.S. Pat. No. 6,242,258 to Haselton and Alexander (Jun. 5, 2001) entitled “Methods for the selective regulation of DNA and RNA transcription and translation by photoactivation”; U.S. Pat. No. 6,500,615; U.S. Pat. No. 6,498,035; U.S. Pat. No. 6,395,544; U.S. Pat. No. 5,563,050; E. Schuch et al (1991) Symp Soc. Exp Biol 45:117-127; de Lange et al., (1995) Curr Top Microbiol Immunol 197:57-75; Hamilton et al. (1995) Curr Top Microbiol Immunol 197:77-89; Finnegan et al., (1996) Proc Natl Acad Sci USA 93:8449-8454; Uhlmann and A. Pepan (1990), Chem. Rev. 90:543; P. D. Cook (1991), Anti-Cancer Drug Design 6:585; J. Goodchild, Bioconjugate Chem. 1 (1990) 165; and, S. L. Beaucage and R. P. Iyer (1993), Tetrahedron 49:6123; and F. Eckstein, Ed. (1991), Oligonucleotides and Analogues—A Practical Approach, IRL Press.

Gene expression can also be inhibited by RNA silencing or interference. “RNA silencing” refers to any mechanism through which the presence of a single-stranded or, typically, a double-stranded RNA in a cell results in inhibition of expression of a target gene comprising a sequence identical or nearly identical to that of the RNA, including, but not limited to, RNA interference, repression of translation of a target mRNA transcribed from the target gene without alteration of the mRNA's stability, and transcriptional silencing (e.g., histone acetylation and heterochromatin formation leading to inhibition of transcription of the target mRNA).

The term “RNA interference” (“RNAi,” sometimes called RNA-mediated interference, post-transcriptional gene silencing, or quelling) refers to a phenomenon in which the presence of RNA, typically double-stranded RNA, in a cell results in inhibition of expression of a gene comprising a sequence identical, or nearly identical, to that of the double-stranded RNA. The double-stranded RNA responsible for inducing RNAi is called an “interfering RNA.” Expression of the gene is inhibited by the mechanism of RNAi as described below, in which the presence of the interfering RNA results in degradation of mRNA transcribed from the gene and thus in decreased levels of the mRNA and any encoded protein. For example, RNAi was used to knock out expression of pha-4 in the present invention to show the importance of pha-4 in dietary restriction induced longevity and could be used to reduce or knock-out expression of daf-16.

The mechanism of RNAi has been and is being extensively investigated in a number of eukaryotic organisms and cell types. See, for example, the following reviews: McManus and Sharp (2002) “Gene silencing in mammals by small interfering RNAs” Nature Reviews Genetics 3:737-747; Hutvagner and Zamore (2002) “RNAi: Nature abhors a double strand” Curr Opin Genet & Dev 200:225-232; Hannon (2002) “RNA interference” Nature 418:244-251; Agami (2002) “RNAi and related mechanisms and their potential use for therapy” Curr Opin Chem Biol 6:829-834; Tuschl and Borkhardt (2002) “Small interfering RNAs: A revolutionary tool for the analysis of gene function and gene therapy” Molecular Interventions 2:158-167; Nishikura (2001) “A short primer on RNAi: RNA-directed RNA polymerase acts as a key catalyst” Cell 107:415-418; and Zamore (2001) “RNA interference: Listening to the sound of silence” Nature Structural Biology 8:746-750. RNAi is also described in the patent literature; see, e.g., CA 2359180 by Kreutzer and Limmer entitled “Method and medicament for inhibiting the expression of a given gene”; WO 01/68836 by Beach et al. entitled “Methods and compositions for RNA interference”; WO 01/70949 by Graham et al. entitled “Genetic silencing”; and WO 01/75164 by Tuschl et al. entitled “RNA sequence-specific mediators of RNA interference.”

In brief, double-stranded RNA introduced into a cell (e.g., into the cytoplasm) is processed, for example by an RNAse III-like enzyme called Dicer, into shorter double-stranded fragments called small interfering RNAs (siRNAs, also called short interfering RNAs). The length and nature of the siRNAs produced is dependent on the species of the cell, although typically siRNAs are 21-25 nucleotides long (e.g., an siRNA may have a 19 base pair duplex portion with two nucleotide 3′ overhangs at each end). Similar siRNAs can be produced in vitro (e.g., by chemical synthesis or in vitro transcription) and introduced into the cell to induce RNAi. The siRNA becomes associated with an RNA-induced silencing complex (RISC). Separation of the sense and antisense strands of the siRNA, and interaction of the siRNA antisense strand with its target mRNA through complementary base-pairing interactions, optionally occurs. Finally, the mRNA is cleaved and degraded.

Expression of a target gene in a cell can thus be specifically inhibited by introducing an appropriately chosen double-stranded RNA into the cell. Guidelines for design of suitable interfering RNAs are known to those of skill in the art. For example, interfering RNAs are typically designed against exon sequences, rather than introns or untranslated regions. Characteristics of high efficiency interfering RNAs may vary by cell type. For example, although siRNAs may require 3′ overhangs and 5′ phosphates for most efficient induction of RNAi in Drosophila cells, in mammalian cells blunt ended siRNAs and/or RNAs lacking 5′ phosphates can induce RNAi as effectively as siRNAs with 3′ overhangs and/or 5′ phosphates (see, e.g., Czaudema et al. (2003) “Structural variations and stabilizing modifications of synthetic siRNAs in mammalian cells” Nucl Acids Res 31:2705-2716). As another example, since double-stranded RNAs greater than 30-80 base pairs long activate the antiviral interferon response in mammalian cells and result in non-specific silencing, interfering RNAs for use in mammalian cells are typically less than 30 base pairs (for example, Caplen et al. (2001) “Specific inhibition of gene expression by small double-stranded RNAs in invertebrate and vertebrate systems” Proc. Natl. Acad. Sci. USA 98:9742-9747, Elbashir et al. (2001) “Duplexes of 21-nucleotide RNAs mediate RNA interference in cultured mammalian cells” Nature 411:494-498 and Elbashir et al. (2002) “Analysis of gene function in somatic mammalian cells using small interfering RNAs” Methods 26:199-213 describe the use of 21 nucleotide siRNAs to specifically inhibit gene expression in mammalian cell lines, and Kim et al. (2005) “Synthetic dsRNA Dicer substrates enhance RNAi potency and efficacy” Nature Biotechnology 23:222-226 describes use of 25-30 nucleotide duplexes). The sense and antisense strands of a siRNA are typically, but not necessarily, completely complementary to each other over the double-stranded region of the siRNA (excluding any overhangs). The antisense strand is typically completely complementary to the target mRNA over the same region, although some nucleotide substitutions can be tolerated (e.g., a one or two nucleotide mismatch between the antisense strand and the mRNA can still result in RNAi, although at reduced efficiency). The ends of the double-stranded region are typically more tolerant to substitution than the middle; for example, as little as 15 bp (base pairs) of complementarity between the antisense strand and the target mRNA in the context of a 21 mer with a 19 bp double-stranded region has been shown to result in a functional siRNA (see, e.g., Czauderna et al. (2003) “Structural variations and stabilizing modifications of synthetic siRNAs in mammalian cells” Nucl Acids Res 31:2705-2716). Any overhangs can but need not be complementary to the target mRNA; for example, TT (two 2′-deoxythymidines) overhangs are frequently used to reduce synthesis costs.

Although double-stranded RNAs (e.g., double-stranded siRNAs) were initially thought to be required to initiate RNAi, several recent reports indicate that the antisense strand of such siRNAs is sufficient to initiate RNAi. Single-stranded antisense siRNAs can initiate RNAi through the same pathway as double-stranded siRNAs (as evidenced, for example, by the appearance of specific mRNA endonucleolytic cleavage fragments). As for double-stranded interfering RNAs, characteristics of high-efficiency single-stranded siRNAs may vary by cell type (e.g., a 5′ phosphate may be required on the antisense strand for efficient induction of RNAi in some cell types, while a free 5′ hydroxyl is sufficient in other cell types capable of phosphorylating the hydroxyl). See, e.g., Martinez et al. (2002) “Single-stranded antisense siRNAs guide target RNA cleavage in RNAi” Cell 110:563-574; Amarzguioui et al. (2003) “Tolerance for mutations and chemical modifications in a siRNA” Nucl. Acids Res. 31:589-595; Holen et al. (2003) “Similar behavior of single-strand and double-strand siRNAs suggests that they act through a common RNAi pathway” Nucl. Acids Res. 31:2401-2407; and Schwarz et al. (2002) Mol. Cell. 10:537-548.

Due to currently unexplained differences in efficiency between siRNAs corresponding to different regions of a given target mRNA, several siRNAs are typically designed and tested against the target mRNA to determine which siRNA is most effective. Interfering RNAs can also be produced as small hairpin RNAs (shRNAs, also called short hairpin RNAs), which are processed in the cell into siRNA-like molecules that initiate RNAi (see, e.g., Siolas et al. (2005) “Synthetic shRNAs as potent RNAi triggers” Nature Biotechnology 23:227-231).

The presence of RNA, particularly double-stranded RNA, in a cell can result in inhibition of expression of a gene comprising a sequence identical or nearly identical to that of the RNA through mechanisms other than RNAi. For example, double-stranded RNAs that are partially complementary to a target mRNA can repress translation of the mRNA without affecting its stability. As another example, double-stranded RNAs can induce histone methylation and heterochromatin formation, leading to transcriptional silencing of a gene comprising a sequence identical or nearly identical to that of the RNA (see, e.g., Schramke and Allshire (2003) “Hairpin RNAs and retrotransposon LTRs effect RNAi and chromatin-based gene silencing” Science 301:1069-1074; Kawasaki and Taira (2004) “Induction of DNA methylation and gene silencing by short interfering RNAs in human cells” Nature 431:211-217; and Morris et al. (2004) “Small interfering RNA-induced transcriptional gene silencing in human cells” Science 305:1289-1292).

Short RNAs called microRNAs (miRNAs) have been identified in a variety of species. Typically, these endogenous RNAs are each transcribed as a long RNA and then processed to a pre-miRNA of approximately 60-75 nucleotides that forms an imperfect hairpin (stem-loop) structure. The pre-miRNA is typically then cleaved, e.g., by Dicer, to form the mature miRNA. Mature miRNAs are typically approximately 21-25 nucleotides in length, but can vary, e.g., from about 14 to about 25 or more nucleotides. Some, though not all, miRNAs have been shown to inhibit translation of mRNAs bearing partially complementary sequences. Such miRNAs contain one or more internal mismatches to the corresponding mRNA that are predicted to result in a bulge in the center of the duplex formed by the binding of the miRNA antisense strand to the mRNA. The miRNA typically forms approximately 14-17 Watson-Crick base pairs with the mRNA; additional wobble base pairs can also be formed. In addition, short synthetic double-stranded RNAs (e.g., similar to siRNAs) containing central mismatches to the corresponding mRNA have been shown to repress translation (but not initiate degradation) of the mRNA. See, for example, Zeng et al. (2003) “MicroRNAs and small interfering RNAs can inhibit mRNA expression by similar mechanisms” Proc. Natl. Acad. Sci. USA 100:9779-9784; Doench et al. (2003) “siRNAs can function as miRNAs” Genes & Dev. 17:438-442; Bartel and Bartel (2003) “MicroRNAs: At the root of plant development?” Plant Physiology 132:709-717; Schwarz and Zamore (2002) “Why do miRNAs live in the miRNP?” Genes & Dev. 16:1025-1031; Tang et al. (2003) “A biochemical framework for RNA silencing in plants” Genes & Dev. 17:49-63; Meister et al. (2004) “Sequence-specific inhibition of microRNA- and siRNA-induced RNA silencing” RNA 10:544-550; Nelson et al. (2003) “The microRNA world: Small is mighty” Trends Biochem. Sci. 28:534-540; Scacheri et al. (2004) “Short interfering RNAs can induce unexpected and divergent changes in the levels of untargeted proteins in mammalian cells” Proc. Natl. Acad. Sci. USA 101: 1892-1897; Sempere et al. (2004) “Expression profiling of mammalian microRNAs uncovers a subset of brain-expressed microRNAs with possible roles in murine and human neuronal differentiation” Genome Biology 5:R13; Dykxhoorn et al. (2003) “Killing the messenger: Short RNAs that silence gene expression” Nature Reviews Molec. and Cell Biol. 4:457-467; McManus (2003) “MicroRNAs and cancer” Semin Cancer Biol. 13:253-288; and Stark et al. (2003) “Identification of Drosophila microRNA targets” PLoS Biol. 1:E60.

The cellular machinery involved in translational repression of mRNAs by partially complementary RNAs (e.g., certain miRNAs) appears to partially overlap that involved in RNAi, although, as noted, translation of the mRNAs, not their stability, is affected and the mRNAs are typically not degraded.

The location and/or size of the bulge(s) formed when the antisense strand of the RNA binds the mRNA can affect the ability of the RNA to repress translation of the mRNA. Similarly, location and/or size of any bulges within the RNA itself can also affect efficiency of translational repression. See, e.g., the references above. Typically, translational repression is most effective when the antisense strand of the RNA is complementary to the 3′ untranslated region (3′ UTR) of the mRNA. Multiple repeats, e.g., tandem repeats, of the sequence complementary to the antisense strand of the RNA can also provide more effective translational repression; for example, some mRNAs that are translationally repressed by endogenous miRNAs contain 7-8 repeats of the miRNA binding sequence at their 3′ UTRs. It is worth noting that translational repression appears to be more dependent on concentration of the RNA than RNA interference does; translational repression is thought to involve binding of a single mRNA by each repressing RNA, while RNAi is thought to involve cleavage of multiple copies of the mRNA by a single siRNA-RISC complex.

Guidance for design of a suitable RNA to repress translation of a given target mRNA can be found in the literature (e.g., the references above and Doench and Sharp (2004) “Specificity of microRNA target selection in translational repression” Genes & Dev. 18:504-511; Rehmsmeier et al. (2004) “Fast and effective prediction of microRNA/target duplexes” RNA 10:1507-1517; Robins et al. (2005) “Incorporating structure to predict microRNA targets” Proc Natl Acad Sci 102:4006-4009; and Mattick and Makunin (2005) “Small regulatory RNAs in mammals” Hum. Mol. Genet. 14:R121-R132, among many others) and herein. However, due to differences in efficiency of translational repression between RNAs of different structure (e.g., bulge size, sequence, and/or location) and RNAs corresponding to different regions of the target mRNA, several RNAs are optionally designed and tested against a target mRNA, e.g., pha-4 or daf-16 mRNA, to determine which is most effective at repressing translation of the target mRNA.

### Making Knock-Out Animals and Transgenics

In another aspect, the invention includes knock out and/or transgenic animals. For example, non-human laboratory animals that comprise a knock out in an endogenous pha-4 and/or daf-16 gene can be made and can additionally include a heterologous pha-4 or daf-16 homolog gene (e.g., from a human source) corresponding to the knock out. For example, nematodes for longevity modulator screening can be made to express one or more of the human foxa genes.

A transgenic animal is typically an animal that has had DNA introduced into one or more of its cells artificially. This is most commonly done in one of two ways. First, DNA can be integrated randomly by injecting it into the pronucleus of a fertilized ovum. In this case, the DNA can integrate anywhere in the genome. In this approach, there is no need for homology between the injected DNA and the host genome. Second, targeted insertion can be accomplished by introducing heterologous DNA into embryonic stem (ES) cells and selecting for cells in which the heterologous DNA has undergone homologous recombination with homologous sequences of the cellular genome. Typically, there are several kilobases of homology between the heterologous and genomic DNA, and positive selectable markers (e.g., antibiotic resistance genes) are included in the heterologous DNA to provide for selection of transformants. In addition, negative selectable markers (e.g., “toxic” genes such as barnase) can be used to select against cells that have incorporated DNA by non-homologous recombination (i.e., random insertion).

One common use of targeted insertion of DNA is to make knock-out mice. Mice provide a very useful laboratory animal, due to the ease with which the animals can be bread, made recombinant, etc. Typically, when making knock outs in mice (or other laboratory animals such as flies or nemotodes), homologous recombination is used to insert a selectable gene driven by a constitutive promoter into an essential exon of the gene that one wishes to disrupt (e.g., the first coding exon). To accomplish this, the selectable marker is flanked by large stretches of DNA that match the genomic sequences surrounding the desired insertion point. Once this construct is electroporated into ES cells, the cells' own machinery performs the homologous recombination. To make it possible to select against ES cells that incorporate DNA by non-homologous recombination, it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring.

Transgenic animals are a useful tool for studying gene function and testing modulators. Human (or other selected) pha-4 or daf-16 homolog genes can be introduced in place of endogenous pha-4 or daf-16 genes of a laboratory animal, making it possible to study function of the human (or other) polypeptide or complex in the easily manipulated and studied laboratory animal. It will be appreciated that there is not precise correspondence between protein structure or function of different animals, making the ability to study the human or other gene of interest particularly useful when developing clinical candidate modulators. Although similar genetic manipulations can be performed in tissue culture, the interaction of pha-4 and daf-16 in the context of an intact organism provides a more complete and physiologically relevant picture of function than could be achieved in non-cell based assays or simple cell-based screening assays. Accordingly, knock-out transgenic animals are particularly useful when analyzing modulators identified in high throughput in vitro (e.g., cell-free and/or cell-based) systems.

An example of a preferred animal model is a nematode that expresses pha-4 or a nematode or other animal with the endogenous pha-4 gene replaced with the corresponding human gene, e.g., a foxa gene. Another model is optionally transformed with a foxa gene and has a knock out gene for any genes orthologous to daf-16. In particular, the assays detect, for example, the presence of increased or decreased activity or expression of pha-4 or a homolog thereof (such as a foxa gene from a human or other animal), e.g., on the basis of increased or decreased pha-4 mRNA expression, increased or decreased levels of pha-4 protein products, or increased or decreased levels of expression of a marker gene (e.g., beta-galactosidase, green fluorescent protein, alkaline phosphatase or luciferase), e.g., operably joined to an pha-4 regulatory region in a recombinant construct, increased or decreased expression of daf-16, increased or decreased expression of the sod genes. Cells known to express a pha-4, or transformed to express a particular pha-4 or daf-16 homolog, are incubated and one or more test compounds are added to the medium. In addition, in higher organisms with at least two pha-4 homolog genes, such as humans, compounds that selectively induce or inhibit the activity or expression of one pha-4 protein and not another may be identified in such assays. Such assays, for example, use pairs of cell-lines, each only expressing one pha-4 homolog gene and comparing the effect of the compound on each cell-line. After allowing a sufficient period of time (e.g., 0-72 hours) for the compound to induce or inhibit the activity or expression of pha-4, any change in levels of activity or expression from an established baseline may be detected using any of the techniques described above.

### Further Details Regarding Nucleic Acid and Polypeptide Sequences and Variants

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). For example, foxa1, foxa2, and foxa3 are homologous, e.g., 91% similar and 85% identical, to pha-4, e.g., as shown in FIG. 6. The precise percentage of similarity between sequences that is useful in establishing homology varies with the nucleic acid and protein at issue, but as little as 25% sequence similarity over 50, 100, 150 or more residues (nucleotides or amino acids) is routinely used to establish homology. Higher levels of sequence similarity, e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are described herein and are generally available, and well known to those of skill in the art.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. A typical reference sequence of the invention is optionally a nucleic acid or amino acid sequence corresponding to pha-4 or daf-16.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), Clustl W, or by visual inspection (see generally Current Protocols in Molecular Biology, Ausubel et al., eds., Current Protocols, a joint venture between Greene Publishing Associates, Inc. and John Wiley & Sons, Inc., supplemented through 2006).

One example of an algorithm that is suitable for determining percent sequence identity and sequence similarity, e.g., to determine whether a nucleic acid or polypeptide is a homolog of pha-4, foxa1, foxa2, foxa3, or daf-16, is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5,

N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in identifying homologous sequences for use in the methods described herein.

In the example shown in FIG. 6, the homology or percent similarity between pha-4 transcription factor protein and the foxa transcription factors is illustrated.

### Further Details Regarding Antibodies

In another aspect, antibodies to pha-4 and/or daf-16 polypeptides (or complexes thereof) can be generated using methods that are well known. The antibodies can be utilized for detecting and/or purifying polypeptides or complexes of interest. Antibodies can optionally discriminate the polypeptides from homologues, and/or can be used in biosensor applications. Antibodies can also be used to block or enhance function of the polypeptides and complexes, in vivo, in situ or in vitro. Thus, antibodies to pha-4 and/or daf-16 and or a complex thereof can be used as therapeutic reagents. As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein.

For the production of antibodies to a polypeptide encoded by a sequence of interest, e.g., pha-4, daf-16, and/or a foxa gene, or conservative variant or fragment thereof, various host animals may be immunized by injection with the polypeptide, or a portion thereof. Such host animals may include, but are not limited to, rabbits, mice and rats, and the like. Various adjuvants may be used to enhance the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler and Milstein (Nature 256:495-497, 1975; and U.S. Pat. No. 4,376,110), the human B-cell hybridoma technique (Kosbor et al., Immunology Today 4:72, 1983; Cole et al., Proc. Nat'l. Acad. Sci. USA 80:2026-2030, 1983), and the EBV-hybridoma technique (Cole et al., Monoclonal Antibodies and Cancer Therapy, Alan R. Liss, Inc., pp. 77-96, 1985). Such antibodies may be of any immunoglobulin class, including IgG, IgM, IgE, IgA, IgD, and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., Proc. Nat'l. Acad. Sci. USA 81:6851-6855, 1984; Neuberger et al., Nature 312:604-608, 1984; Takeda et al., Nature 314:452-454, 1985) by splicing the genes from a mouse antibody molecule of appropriate antigen specificity, together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Alternatively, techniques described for the production of single-chain antibodies (U.S. Pat. No. 4,946,778; Bird, Science 242:423-426, 1988; Huston et al., Proc. Nat'l. Acad. Sci. USA 85:5879-5883, 1988; and Ward et al., Nature 334:544-546, 1989) can be adapted to produce differentially expressed gene-single chain antibodies. Single chain antibodies are formed by linking the heavy and light chain fragments of the Fv region via an amino acid bridge, resulting in a single-chain polypeptide.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science 246:1275-1281, 1989) to allow rapid and easy identification of monoclonal Fab fragments with the desired specificity.

The protocols for detecting and measuring the expression of the described polypeptides herein, using the above mentioned antibodies, are well known in the art. Such methods include, but are not limited to, dot blotting, western blotting, competitive and noncompetitive protein binding assays, enzyme-linked immunosorbant assays (ELISA), immunohistochemistry, fluorescence-activated cell sorting (FACS), and others commonly used and widely described in scientific and patent literature, and many employed commercially.

One method, for ease of detection, is the sandwich ELISA, of which a number of variations exist, all of which are intended to be encompassed by the present invention. For example, in a typical forward assay, unlabeled antibody is immobilized on a solid substrate and the sample to be tested is brought into contact with the bound molecule and incubated for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen, e.g., a pha-4 or daf-16 polypeptide, is determined by observation of a signal, or may be quantitated by comparing with a control sample containing known amounts of antigen. Variations on the forward assay include the simultaneous assay, in which both sample and antibody are added simultaneously to the bound antibody, or a reverse assay, in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody that is specific for the protein expressed by the gene of interest, e.g., pha-4.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of antigen present in a sample.

Alternately, fluorescent compounds, such as fluorescein and rhodamine, can be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Antibodies specific for pha-4 and/or daf-16 are useful in modulating (e.g., increasing) lifespan, as well as in targeting cells that express pha-4 and/or daf-16. In human therapeutic applications of such antibodies, e.g., where an increase in longevity is desired, including any of those applications noted herein, antibodies will normally be humanized before use. Thus, antibodies to pha-4 and/or daf-16 can be generated by any available method as noted above, and subsequently humanized appropriately for use in vivo in humans. Many methods of humanizing antibodies are currently available, including those described in Howard and Kaser Making and Using Antibodies: A Practical Handbook ISBN: 0849335280 (2006). In typical approaches, humanized Abs are created by combining, at the genetic level, the complementarity-determining regions of a murine (or other mammalian) mAb with the framework sequences of a human Ab variable domain. This leads to a functional Ab with reduced immunogenic side effects in human therapy. Such techniques are generally described in in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429. Methods of making “superhumanized” antibodies with still further reduced immunogenicity in humans are described in Tan et al. (2002) ““Superhumanized” Antibodies: Reduction of Immunogenic Potential by Complementarity-Determining Region Grafting with Human Germline Sequences: Application to an Anti-CD28,” The Journal of Immunology, 169:1119-1125. Any available humanization method can be applied to making humanized antibodies of the present invention.

### Modulating Longevity Using Compounds of the Invention

Compounds identified using the screening assays and systems described above or any other compounds that modulate longevity, e.g., through the dietary restriction pathway based on pha-4, are another embodiment of the invention. The compounds typically modulate, e.g., increase, expression or activity of pha-4 and optionally also modulate, e.g., decrease, expression of daf-16. In addition, the compounds optionally modulate the expression or activity of one or more member of the sod gene family, e.g., sod-2 and/or sod-4, which are regulated by pha-4, or sod-1 and/or sod-5, which are regulated by both pha-4 and daf-16. In one preferred embodiment, a compound is provided that increases expression of pha-4 and decreases expression of daf-16.

The compounds used to modulate longevity in a subject, e.g., a mammal such as a human, are optionally any of the compounds discussed above that are optionally used as test compounds in the screening methods of the invention. A combination of compounds is also optionally used to modulate longevity, e.g., increase lifespan. For example, a first compound is optionally used to increase expression of pha-4 and a second compound is optionally used to decrease expression of daf-16. The compounds can be administered together in a single pharmaceutical formulation or as two separate pharmaceutical formulations.

The compounds of the invention are typically administered to a subject or patient, e.g., to increase longevity, to treat premature aging, delay the onset of age-related diseases, such as some cancers, or enhance quality of life during the later part of a subject's lifespan, e.g., by preventing or alleviating symptoms of aging such as cognitive and motor deficits. The subject is optionally treated once with a compound that acts over an extended period of time or given daily or monthly doses or the like for a shorter acting compound. In addition, a subject can be subjected to dietary restriction while on a pharmaceutical dosing regimen to extend longevity or the compound used can also act in the absence of any dietary restrictions.

Pharmaceutically Acceptable Modulators of Longevity

Therapeutic formulations of the compounds of the invention (e.g., pha-4 expression modulators, such as ones identified as described herein) used in accordance with the present invention are prepared for storage by mixing a moiety having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as Tween®, Pluronics®, or PEG, and the like.

Lyophilized formulations adapted for subcutaneous administration are described, for example, in U.S. Pat. No. 6,267,958 (Andya et al.). Such lyophilized formulations may be reconstituted with a suitable diluent to a high concentration and the reconstituted formulation may be administered subcutaneously to a subject to be treated as described herein. Crystallized forms of the moiety are also contemplated. See, for example, U.S. 2002/0136719A1 (Shenoy et al.).

The active ingredients, e.g., one or more longevity modulators, may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug-delivery systems (for example, liposomes, albumin microspheres, microemulsions, nano-particles and nanocapsules) or in macroemulsions. Such techniques are disclosed, e.g., in Remington's Pharmaceutical Sciences 16th edition, Osol, A. Ed. (1980).

Sustained-release preparations may also be prepared. Suitable examples of sustained-release preparations include semipermeable matrices of solid hydrophobic polymers containing an active ingredient, e.g., an agonistic antibody to pha-4, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid.

The formulations to be used for in vivo administration are typically sterile. This is readily accomplished by filtration through sterile filtration membranes.

Administration of Pharmaceutical Acceptable Longevity Modulators

As will be understood by those of ordinary skill in the art, the appropriate doses of compounds of the invention (e.g., polypeptides, antisense, RNAi molecules, antibodies, etc.) will be generally around those already employed in clinical therapies wherein similar moieties are administered alone or in combination with other therapeutics. The physician administering treatment will be able to determine the appropriate dose for the individual subject. Preparation and dosing schedules for commercially available second compounds administered in combination with the moieties may be used according to manufacturers' instructions or determined empirically by the skilled practitioner.

For the prevention or treatment of disease, the appropriate dosage of a modulator, e.g., identified by the methods provided herein, will depend on the type of disease to be treated, e.g., premature aging or extension of lifespan in a mature adult, the severity and course of the disease, whether the modulator is administered for preventive or therapeutic purposes, previous therapy, the patient's clinical history, and the discretion of the attending physician. The compound or combination of compounds is suitably administered to the patient in one dose or more typically over a series of treatments.

Depending on the type and severity of the disease, about 1 μg/kg to 50 mg/kg (e.g. 0.1-20 mg/kg) of the moiety is an initial candidate dosage for administration to a patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of symptoms occurs, e.g., age related symptoms. However, other dosage regimens may be useful. Typically, a clinician will administer a moiety of the invention (alone or in combination with a second compound) until a dosage(s) is reached that provides the required biological effect, e.g., a delay in aging or a halt in premature aging. The progress of the therapy of the invention is easily monitored by conventional techniques and assays.

Compounds that increase longevity and/or delay or halt the onset of age-related diseases or conditions can be administered by any suitable means, including parenteral, topical, subcutaneous, intraperitoneal, intrapulmonary, intranasal, and/or intralesional administration. Parenteral infusions include intramuscular, intravenous, intraarterial, intraperitoneal, or subcutaneous administration.

Additional Details Regarding Treatment to Modulate Longevity

The term “patient” or “subject” as used herein refers to any individual to which the methods of treatment are performed, e.g., a person receiving treatment to delay or halt aging. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician. In the present invention, the medical response is optionally a longer lifespan, or a delay or halt in aging or age-related diseases or conditions.

The term “pharmaceutically acceptable”, when used in reference to a carrier, is meant that the carrier, diluent or excipient must be compatible with the other ingredients of the formulation and not deleterious to the recipient thereof.

The terms “administration” or “administering” is defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The efficacy of a therapeutic method of the invention over time can be identified by an absence of symptoms or clinical signs, e.g., by delayed onset of age-related conditions and lifespan extension, e.g., beyond an average age for a particular population.

Therapeutic Applications

In one aspect, the invention includes rescue of a cell that is defective in function of one or more endogenous foxa or pha-4 genes or polypeptides. This can be accomplished simply by introducing a new copy of the gene (or a heterologous nucleic acid that expresses the relevant protein) into a cell. Other approaches, such as homologous recombination to repair the defective gene (e.g., via chimeraplasty) can also be performed. In any event, rescue of function can be measured, e.g., in any of the assays noted herein. Indeed, this can be used as a general method of screening cells in vitro for activity. Accordingly, in vitro rescue of function is useful in this context for the myriad in vitro screening methods noted above, e.g., for the identification of modulators in cells. The cells that are rescued can include cells in culture, (including primary or secondary cell culture from patients, as well as cultures of well-established cells). Where the cells are isolated from a patient, this has additional diagnostic utility in establishing which sequence is defective in a patient, e.g., one experiencing early onset of age related conditions or diseases.

In another aspect, cell rescue occurs in a patient, e.g., a human or veterinary patient, e.g., to increase longevity. Thus, one aspect of the invention is gene therapy to extend longevity, e.g., in human or veterinary subjects. In these applications, the nucleic acids of the invention are optionally cloned into appropriate gene therapy vectors (and/or are simply delivered as naked or liposome-conjugated nucleic acids), which are then delivered, optionally in combination with appropriate carriers or delivery agents. Proteins can also be delivered directly, but delivery of the nucleic acid is typically preferred in applications where stable expression is desired.

Vectors for administration typically comprise pha-4, foxa, and/or daft 6 genes under the control of a promoter that is expressed in a cell of interest. These can include native pha-4 and/or daf-16 promoters and/or upstream regulatory elements, or other cell specific promoters known to those of skill in the art.

Compositions for administration typically comprise a therapeutically effective amount of the gene therapy vector or other relevant nucleic acid, and a pharmaceutically acceptable carrier or excipient. Such a carrier or excipient includes, but is not limited to, saline, buffered saline, dextrose, water, glycerol, ethanol, and/or combinations thereof. The formulation is made to suit the mode of administration. In general, methods of administering gene therapy vectors for topical use are well known in the art and can be applied to administration of the nucleic acids of the invention, e.g., pha-4 or foxa nucleic acids.

Therapeutic compositions comprising one or more nucleic acid of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

Administration is by any of the routes normally used for introducing a molecule into ultimate contact with cells of interest. Practitioners can select an administration route of interest based on the cell target. Suitable methods of administering such nucleic acids are available and known to those of skill in the art, and, although more than one route can be used to administer a particular composition, a particular route can often provide a more immediate and more effective action or reaction than another route.

The nucleic acids of the invention are administered in any suitable manner, optionally with one or more pharmaceutically acceptable carriers. Pharmaceutically acceptable carriers are determined in part by the particular composition being administered, as well as by the particular method used to administer the composition. Accordingly, there is a wide variety of suitable pharmaceutical formulations for compositions of the present invention, e.g., compositions to increase longevity. Compositions can be administered by a number of routes including, but not limited to: oral, intravenous, intraperitoneal, intramuscular, transdermal, subcutaneous, topical, sublingual, spinal, or rectal administration. Compositions can be administered via liposomes (e.g., topically), or via topical delivery of naked DNA or viral vectors. Such administration routes and appropriate formulations for each are generally known to those of skill in the art.

The compositions, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid, such as a foxa nucleic acid, can be presented in unit-dose or multi-dose sealed containers, such as ampules and vials.

The dose administered to a patient, in the context of the present invention, is sufficient to effect a beneficial therapeutic response in the patient over time, e.g., a halt in the progression of one or more age-related diseases and/or conditions. The dose is determined by the efficacy of the particular vector, or other formulation, and the activity, stability or serum half-life of the polypeptide which is expressed, and the condition of the patient, as well as the body weight or surface area of the patient to be treated. The size of the dose is also determined by the existence, nature, and extent of any adverse side-effects that accompany the administration of a particular vector, formulation, or the like in a particular patient. In determining the effective amount of the vector or formulation to be administered in the treatment of disease, a physician evaluates local expression in the tissue or cell of interest, or circulating plasma levels, formulation toxicities, progression of the relevant disease, and/or where relevant, the production of antibodies to proteins encoded by the polynucleotides. The dose administered, e.g., to a 70 kilogram patient, is typically in the range equivalent to dosages of currently-used therapeutic proteins, adjusted for the altered activity or serum half-life of the relevant composition. The vectors of this invention can supplement treatment conditions by any known conventional therapy, e.g., diet restriction.

### Screening Systems and Kits

In other embodiments, the invention provides a kit useful for the methods and modulators described herein. Such kits optionally comprise one or more containers, labels, and instructions, as well as components for assaying and identifying potential modulators of pha-4. For example, a kit optionally contains an array of nematodes in containers along with a module for assaying lifespan of the nematodes when exposed to various test compounds.

In many embodiments, the kits comprise instructions (e.g., typically written instructions) relating to the use of the kit to identify longevity modulators. In some embodiments, the kits comprise a URL address or phone number or the like for users to contact for instructions or further instructions.

### EXAMPLES

The following are examples illustrating the characterization of pha-4 and daf-16 and their role in dietary restriction mediated longevity. It will be appreciated that such descriptions and examples are not necessarily limiting upon the methods, compositions, systems, etc., of the invention. It is understood that examples and embodiments described herein are for illustrative purposes only and that various modifications or changes in light thereof will be suggested to persons skilled in the art and are to be included within the spirit and purview of this application and within the scope of the appended claims.

### Pha-4 is Required for Multiple Forms of Dietary Restriction

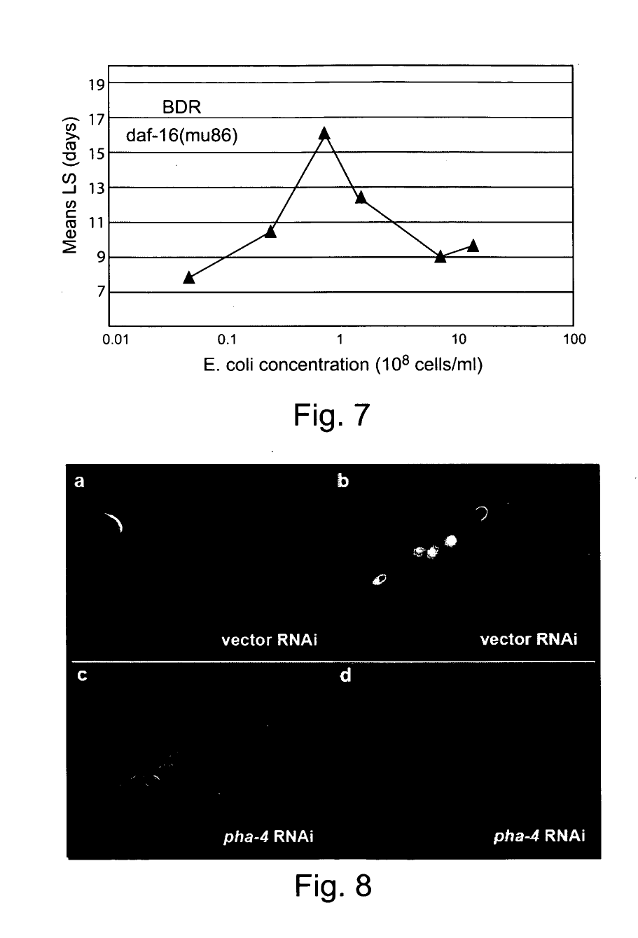

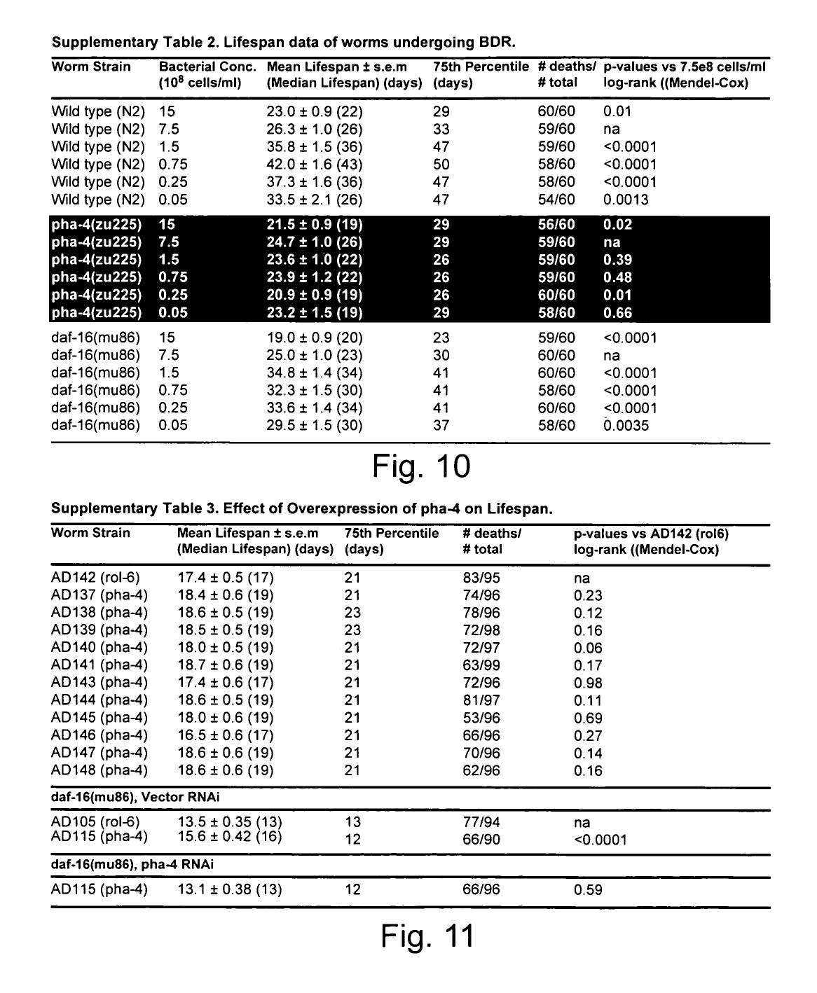

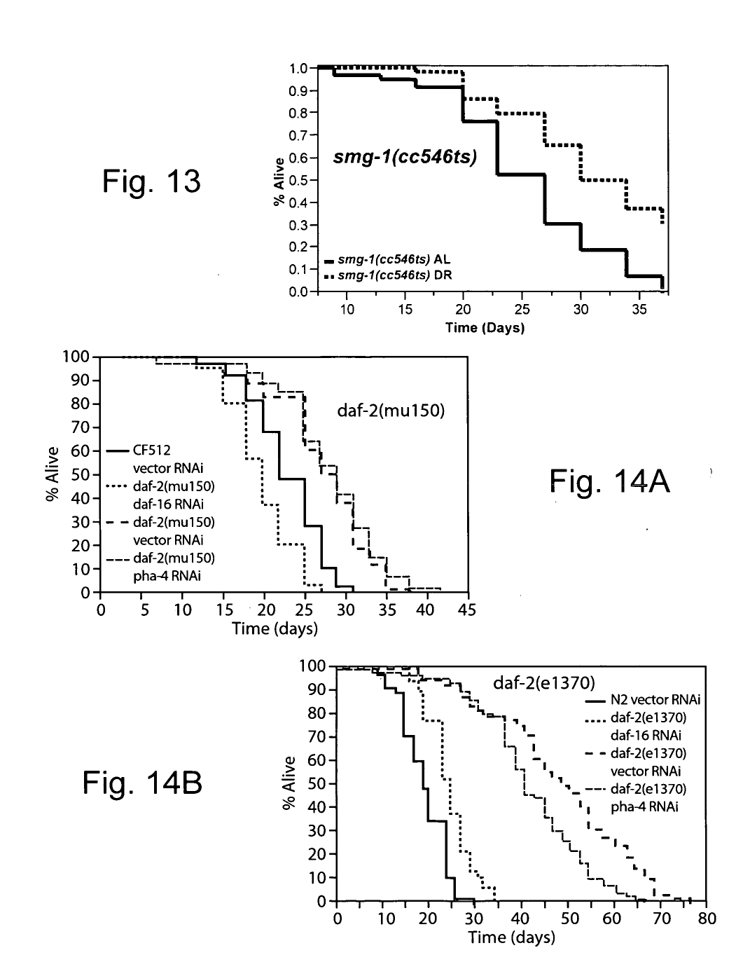

Because pha-4 is required for development of the worm pharynx, and eat-2 mutations affect pharyngeal pumping rates, whether a loss of pha-4 suppressed dietary restriction in a non-genetic model was tested. In the worm, dietary restriction can also be achieved by limiting the concentration of bacteria fed to worms in culture by bacterial dietary restriction (BDR) (See, Klass, Aging in the nematode Caenorhabditis elegans: major biological and environmental factors influencing life span. Mech. Ageing Dev. 6, 413-429 (1977)). At high and extremely low food concentrations, wild-type animals are short lived, whereas conditions of optimal food intake result in increased longevity (see Methods, FIG. 1B and FIG. 10). Much like wild-type animals, and in agreement with previous results (See, Houthoofd, K., et al. Life extension via dietary restriction is independent of the Ins/IGF1 signalling pathway in Caenorhabditis elegans. Exp. Gerontol. 38, 947-954 (2003)), daf-16(mu86)-null mutant animals were longer lived at the optimal concentration and shorter lived at lower and higher concentrations, exhibiting a parabolic curve (FIG. 1B and FIG. 7). In contrast, pha-4(zu225); smg-1(cc546ts) mutant worms, but not smg-1(cc546) control mutant worms (FIG. 13), were short lived at all concentrations and did not exhibit a parabolic curve in response to varying food concentrations. A loss of pha-4 fully blocked the entire response of lifespan to dietary restriction, as would be expected of a gene essential for diet restriction-mediated longevity (FIG. 1B). In all experiments, transfer to restrictive temperatures to inactivate pha-4 as well as dietary restriction treatment itself was delayed until the first day of adulthood to avoid possible developmental abnormalities (see, Gaudet & Mango, Regulation of organogenesis by the Caenorhabditis elegans FoxA protein PHA-4. Science 295, 821-825 (2002). It was also confirmed that a loss of smk-1 using this method suppressed the extended lifespan under conditions of optimal dietary restriction (data not shown).

### Pha-4 is Specific to Diet-Restriction-Induced Longevity

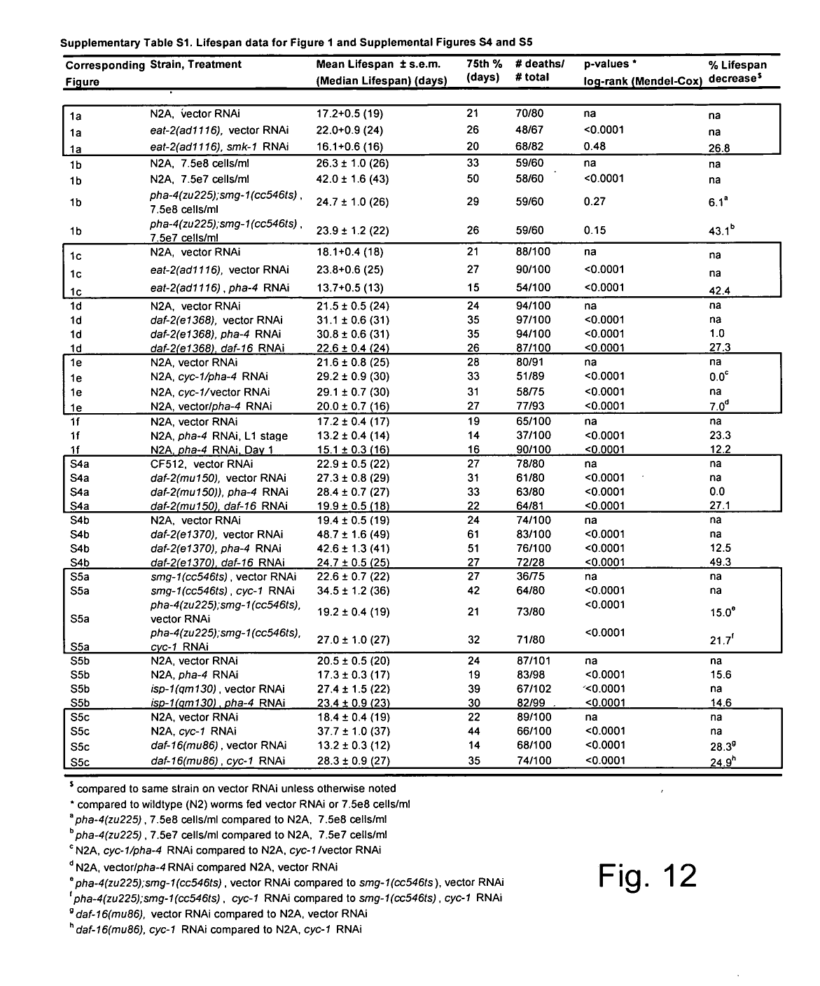

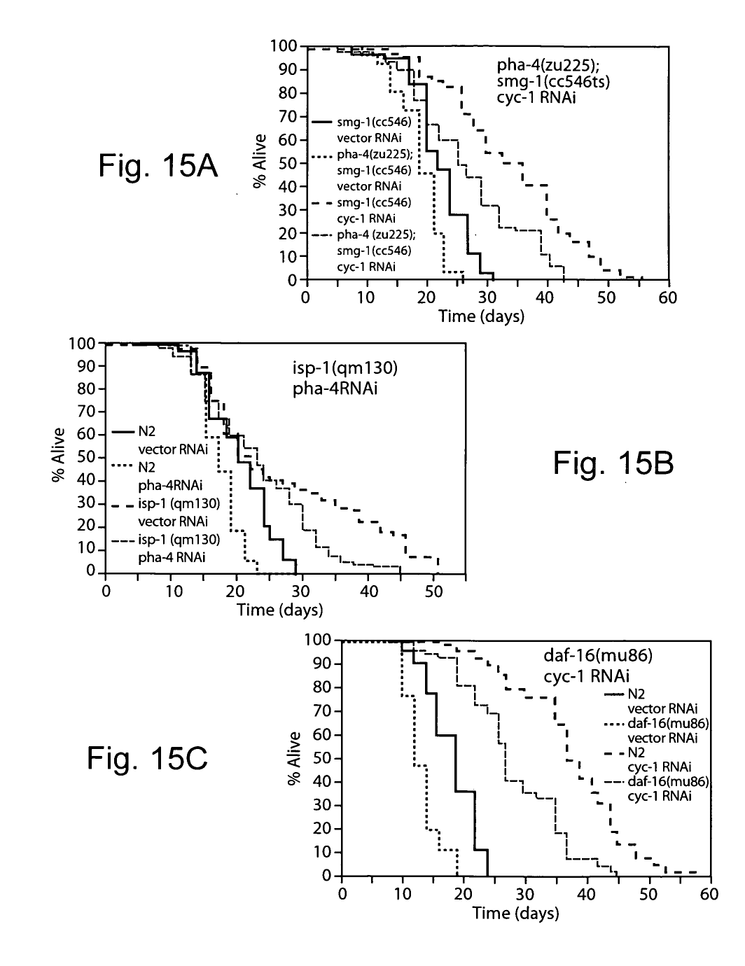

A loss of pha-4 suppressed any potential lifespan extension across a spectrum of bacterial concentrations, suggesting that it was not causing a general sickness in the animal. However, to determine more conclusively whether pha-4 was acting specifically to affect the dietary restriction pathway, its effect on other pathways that influence longevity was examined. Reduced IIS, by mutation of the insulin/IGF-1 receptor daf-2 increases longevity. Pha-4 is not required for the long lifespan of daf-2 mutant animals. RNAi knockdown of daf-16, but not pha-4, completely suppressed the long lifespan of daf-2(e1368) (FIG. 1D), daf-2(mu150), and daf-2(e1370) mutant animals (FIGS. 14A and 14B, respectively). Additionally, it was tested whether pha-4 was required for the long lifespan of animals with reduced mitochondrial electron transport chain activity. Dillin, A. et al. Rates of behavior and aging specified by mitochondrial function during development. Science 298, 2398-2401 (2002). Lee, S. S. et al. A systematic RNAi screen identifies a critical role for mitochondria in C. elegans longevity. Nature Genet. 33, 40-48 (2003). Neither RNAi of pha-4 nor the pha-4(zu225) smg-1 (cc546ts) allele shortened the long lifespan of cyc-1-RNAi-treated animals (FIG. 1E and FIG. 15A, respectively) or isp-1(qm130) mutant animals (FIG. 15B) any more than reduction of pha-4 in a wild-type background (FIG. 1F and FIG. 12) and to a lesser extent than the loss of daf-16 in cyc-1-RNAi-treated animals (FIG. 15C). Therefore, pha-4 was determined to be a specific requirement in the regulation of longevity in worms undergoing dietary restriction, the loss of which does not simply cause a general sickness.

The Role of pha-4 in Development and Longevity is Separable

pha-4 has an essential early role during embryo development in the morphogenesis of the pharynx, and inactivation of pha-4 up to the first larval stage, L1, can result in lethality (Mango et al., The pha-4 gene is required to generate the pharyngeal primordium of Caenorhabditis elegans. Development 120, 3019-3031 (1994)). The early developmental function of pha-4 can be temporally separated from its role in dietary restriction during adulthood. Eat-2(ad1116) mutant animals were allowed to develop through the larval stages (L1-L4) and grow on normal bacteria and were then shifted on the first day of adulthood to bacteria expressing pha-4 double-stranded-RNA, thereby only inactivating pha-4 during adulthood—long after pharyngeal development had completed (Mango et al., Development 120, 3019-3031 (1994)). RNAi of pha-4 during only adulthood suppressed the long lifespan of eat-2(ad1116) mutant animals to wild-type levels (FIG. 2). In support of these data, pha-4(zu225); smg-1(cc546ts) mutant worms in the BDR experiments were not shifted to the restrictive temperature to inactivate pha-4 (Gaudet & Mango, Regulation of organogenesis by the Caenorhabditis elegans FoxA protein PHA-4. Science 295, 821-825 (2002)) until adulthood. In addition, it was considered whether reduction of pha-4 could suppress diet-restriction-mediated longevity by altering pharyngeal function during adulthood by indirectly affecting the feeding rates of animals and pushing diet-restriction animals towards starvation. This hypothesis was found to be inconsistent with multiple observations. First, the pumping (feeding) rate of wild-type animals grown on control bacteria was very similar to the pumping rate of wild-type animals treated with pha-4 RNAi (wild type treated with vector RNAi, 242610 pumps per min (6 s.d.); wild type treated with pha-4 RNAi, 238.2611.5 pumps per min (6 s.d.)). Additionally, eat-2(ad1116) mutant animals treated with pha-4 RNAi did not exhibit altered feeding rates (eat-2(ad1116) treated with vector RNAi, 50.167.1 pumps per min; eat-2(ad1116) treated with pha-4 RNAi, 48.465.5 pumps per min). It was confirmed that RNAi of pha-4 was activated by the time pumping rates were monitored by following the green fluorescent protein (GFP) signal of pha-4-gfp transgenic animals treated with pha-4 RNAi (see Methods and FIG. 8). In agreement with this observation, pha-4 RNAi did not increase longevity of wild-type animals, as would be expected if feeding rates were reduced⁸. In fact, RNAi of pha-4 slightly shortened wild-type longevity, even when applied specifically to adult animals (FIG. 1F). As noted previously, the pha-4(zu225) mutation did not change the parabolic relationship observed between BDR and longevity: it blocked the entire response (FIG. 1B). Finally, pha-4 RNAi did not further increase the long lifespan of daf-2 mutant animals (FIG. 1D and FIGS. 14A and 14B), as is observed with eat-2; daf-2 mutant animals that live longer than either single mutation (See, Lakowski & Hekimi. The genetics of caloric restriction in Caenorhabditis elegans. Proc. Natl. Acad. Sci. USA 95, 13091-13096 (1998)), and did not enhance the long lifespan of animals treated with RNAi or mutation resulting in reduced electron transport chains (FIG. 1E and FIGS. 15A and 15B).

### Pha-4 Expression is Increased in Response to Dietary Restriction

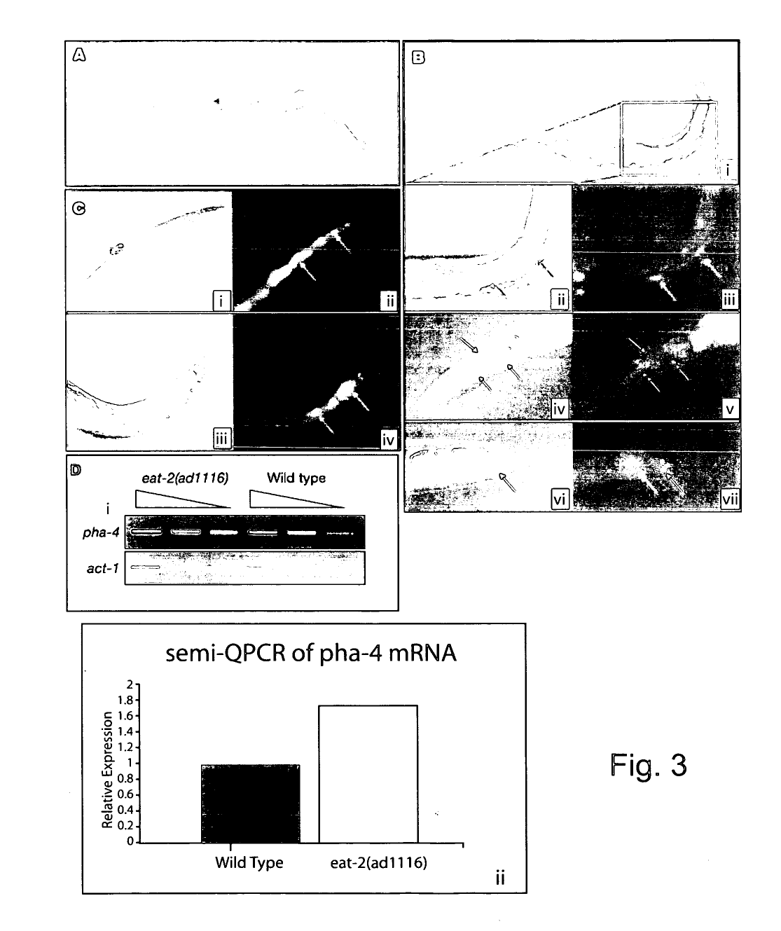

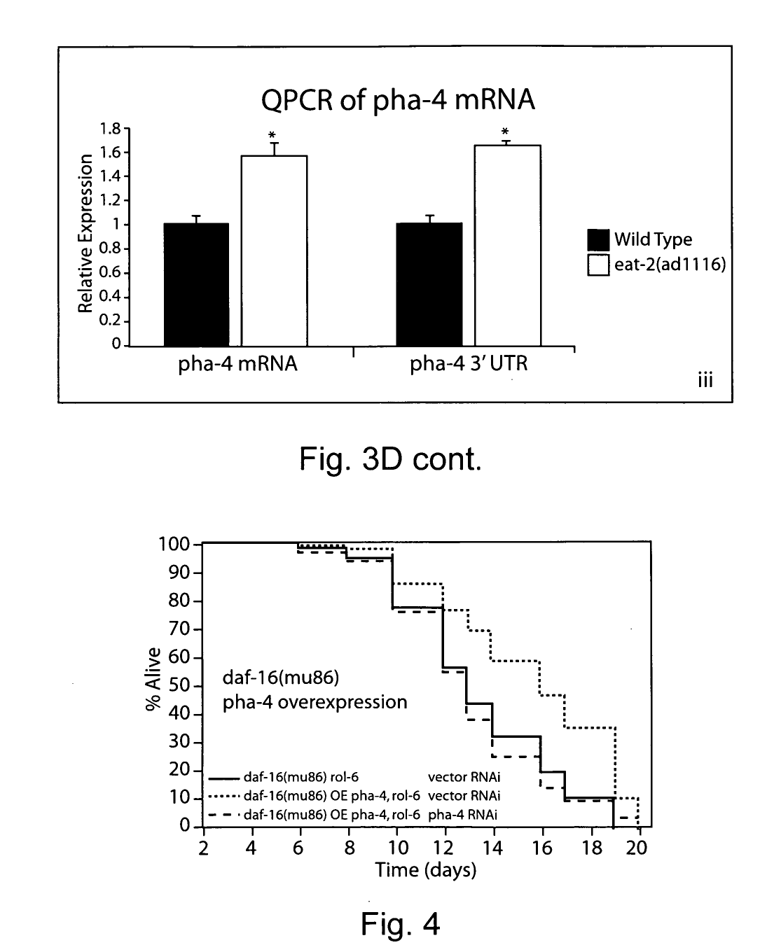

Pha-4 is expressed in the developing pharynx and intestine during embryogenesis and larval stages (Mango et al., Development 120, 3019-3031 (1994); Azzaria et al., A forkhead/HNF-3 homolog expressed in the pharynx and intestine of the Caenorhabditis elegans embryo. Dev. Biol. 178, 289-303 (1996)). The expression pattern of pha-4 during adulthood was examined to determine whether it was different from its developmental expression pattern. Using a red fluorescent protein (RFP) transcriptional fusion to the pha-4 promoter, strong expression was observed in the developing pharynx and in the intestine, as noted previously (Mango et al., Development 120, 3019-3031 (1994); Azzaria, M., et al. Dev. Biol. 178, 289-303 (1996)). In the adult animal, expression was lacking in the pharynx, but still present in the intestine (FIG. 3A). Using a full-length pha-4 complementary DNA translation fusion to GFP under the pha-4 promoter, nuclear localization of PHA-4 was observed during development and adulthood within the same cells (FIG. 3B and see Methods) and pha-4 expression was found in the adult worm to be expanded to a few neuronal cells in the head and tail, which were not found in the developing animal (FIG. 3B). This expression pattern did not change in response to dietary restriction (data not shown), and PHA-4 seemed constitutively nuclear under all conditions tested (FIG. 3C). During embryogenesis, levels of PHA-4 expression determine its binding specificity: low levels of PHA-4 bind high-affinity sites in promoters during early embryogenesis; PHA-4 does not bind to low affinity sites until late in embryogenesis when pha-4 expression levels increase (Gaudet & Mango, Science 295, 821-825 (2002)). Following this paradigm, it was reasoned that expression of pha-4 might increase during dietary restriction to facilitate its binding to diet-restriction-specific genes. Using both semi-quantitative PCR with reverse transcription (RT-PCR) and quantitative real-time RT-PCR (Q-PCR), expression of pha-4 increased by more than 80% in response to dietary restriction (FIG. 3D).

Overexpression of pha-4 Extends Longevity in the Absence of daf-16

Because expression levels of pha-4 were increased in response to dietary restriction, the question of whether overexpression of pha-4 was sufficient to extend longevity under normal feeding conditions was examined. Eleven independent lines overexpressing pha-4 were established (see Methods). In nine lines, pha-4 overexpression increased longevity of wild-type animals, but only slightly (FIG. 11). However, when the same pha-4 expression construct was used to overexpress pha-4 in a daf-16(mu86)-null mutant strain, a statistically significant increase in lifespan was observed (FIG. 4 and FIG. 11). There are at least two explanations for this result. One, an inherent competition between daf-16 and pha-4 in wild-type animals may exist; or two, the role of daf-16 and pha-4 may be partially redundant in determination of longevity of wild-type animals. In any event, the relative increase in lifespan by pha-4 overexpression was greatest in the complete absence of daf-16.

### The sod Gene Family is Differentially Regulated by DAF-16 and PHA-4

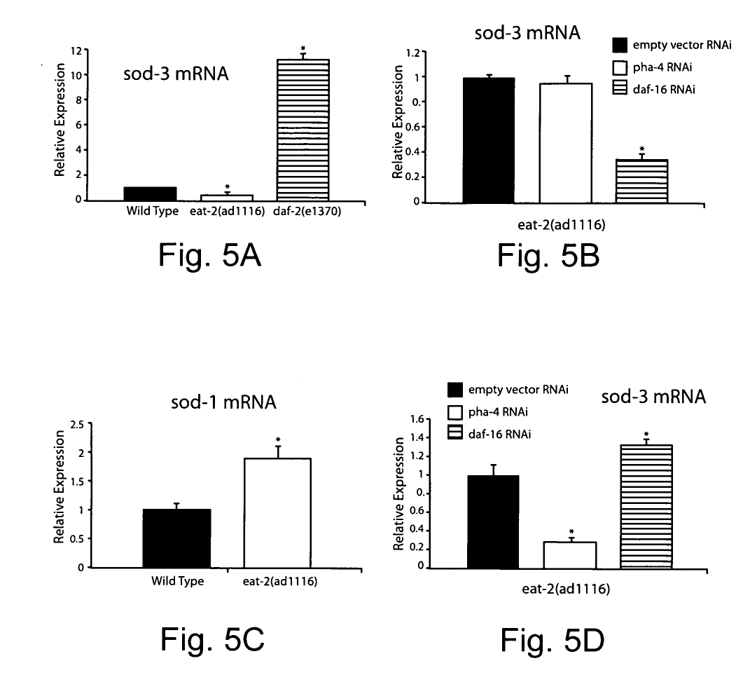

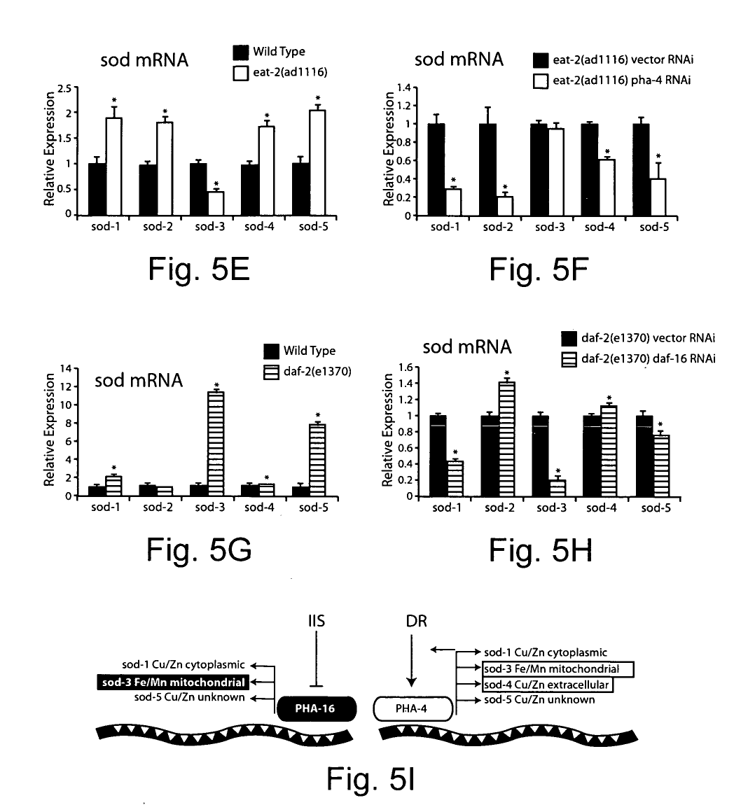

In analysing the potential competition among daf-16 and pha-4, we noticed that the consensus DNA binding sites for DAF-16 and PHA-4 overlap: PHA-4, T(A/G)TT(T/G)(A/G)(T/C) (see, Gaudet & Mango, Science 295, 821-825 (2002)) versus DAF-16, T(A/G)TTTAC (see, Furuyama et al., Identification of the differential distribution patterns of mRNAs and consensus binding sequences for mouse DAF-16 homologues. Biochem. J. 349, 629-634 (2000)). This observation raised the hypothesis that DAF-16 and PHA-4 regulate expression of the same genes either directly or indirectly. sod-3, a mitochondrial Fe/Mn superoxide dismutase (see, Giglio et al., The manganese superoxide dismutase gene of Caenorhabditis elegans. Biochem. Mol. Biol. Int. 33, 37-40 (1994); Hunter et al., J. Biol. Chem. 272, 28652-28659 (1997); Suzuki et al., DNA Res. 3, 171-174 (1996)), is the best characterized DAF-16 target gene and contains three DAF-16 DNA binding sites within the promoter region (See, Honda & Honda. The daf-2 gene network for longevity regulates oxidative stress resistance and Mn-superoxide dismutase gene expression in Caenorhabditis elegans. FASEB J. 13, 1385-1393 (1999)). All three DAF-16 sites overlap with the consensus PHA-4 DNA binding site. Therefore, Q-PCR analysis was used to examine sod-3 expression in diet-restricted, eat-2(ad1116) mutant animals. Surprisingly, no increase in expression levels of sod-3 was found in response to dietary restriction (FIG. 5A, see Methods). Furthermore, the basal level of sod-3 expression was not altered in diet-restricted animals lacking pha-4 (FIG. 5B). The sod-1 promoter contains four consensus PHA-4 binding sites. Furthermore, the mouse sod-1 orthologue, sod-1, has been shown to be a transcriptional target of Foxa1 (See, Carroll, J. S. et al. Chromosome-wide mapping of estrogen receptor binding reveals long-range regulation requiring the forkhead protein FoxA 1. Cell 122, 33-43 (2005)). Sod-1 is a cytoplasmic Cu/Zn superoxide dismutase (See, Giglio et al., The copper/zinc superoxide dismutase gene of Caenorhabditis elegans. Biochem. Mol. Biol. Int. 33, 41-44 (1994)). Whether the C. elegans sod-1 was transcriptionally regulated in eat-2(ad1116) mutant animals was then tested. By Q-PCR analysis, sod-1 expression was greatly upregulated in response to dietary restriction (FIG. 5C). In diet-restricted animals, sod-1 expression was decreased in the absence of pha-4, but was slightly increased in the absence of daf-16 (FIG. 5D). Therefore, expression of sod-1 requires PHA-4, but not DAF-16, in eat-2(ad1116) mutant animals. The C. elegans genome contains five sod genes including sod-3 and sod-1 (C. elegans sequencing consortium. Genome sequence of the nematode C. elegans: a platform for investigating biology. Science 282, 2012-2018 (1998)). The expression patterns of each of the sod gene family members was further investigated under conditions of dietary restriction or reduced IIS signaling. Interestingly, it was found that the expression level of every sod gene except for sod-3 was increased under dietary restriction (FIG. 5E). The increases were pha-4-dependent (FIG. 5F). In response to reduced IIS, sod-1, sod-3 and sod-5 expression levels were increased (FIG. 5G); this increase was daf-16-dependent (FIG. 5H). Therefore, it was determined that sod-2 and sod-4 expression is specific to dietary restriction and dependent on pha-4, whereas sod-3 expression is specific to reduced IIS and dependent on daf-16. Common to both dietary restriction and reduced IIS, expression of sod-1 and sod-5 are increased by PHA-4 and DAF-16, respectively (FIG. 5I). Although each sod gene contains respective predicted DAF-16 and PHA-4 binding sites within their promoters, regulation by additional factors cannot be ruled out at this time.

### Discussion

In worms, pha-4 is bifunctional, having an early developmental function in pharyngeal determination during embryogenesis and the L1 larval stage (see, Gaudet & Mango, Regulation of organogenesis by the Caenorhabditis elegans FoxA protein PHA-4. Science 295, 821-825 (2002); Mango et al., Development 120, 3019-3031 (1994)), and a later function during adulthood in regulating the response to dietary restriction. This dual mode of action of PHA-4 is similar to that of DAF-16, which is required during early larval stages to regulate the dauer developmental decision and reproductive status of the animal, and later during adulthood to regulate the response of ageing to IIS (See, Dillin et al., Timing requirements for insulin/IGF-1 signaling in C. elegans. Science 298, 830-834 (2002)). In mammals, a parallel regulation of insulin levels by FOXO proteins (Puig & Tjian, Transcriptional feedback control of insulin receptor by dFOXO/FOXO1. Genes Dev. 19, 2435-2446 (2005)) and glucagon levels by Foxa1 and Foxa2 (Kaestner et el. Inactivation of the winged helix transcription factor HNF3a affects glucose homeostasis and islet glucagon gene expression in vivo. Genes Dev. 13, 495-504 (1999); Zhang et al. Foxa2 integrates the transcriptional response of the hepatocyte to fasting. Cell Metab. 2, 141-148 (2005)) supports a model in which, under continually low nutrient signaling, PHA-4/Foxa may mediate levels of glucagon or other changes in hormones ultimately capable of regulating the ageing process. In contrast, in times of severe stress or starvation, DAF-16/FOXO will mediate the response to decreased insulin signaling. Although C. elegans does not contain an obvious glucagon orthologue, it does contain a full complement of insulin-like peptides (Pierce et al. Regulation of DAF-2 receptor signaling by human insulin and ins-1, a member of the unusually large and diverse C. elegans insulin gene family. Genes Dev. 15, 672-686 (2001)), suggesting that a conserved functional regulation of glucose homeostasis is present. The finding that some insulin-like peptides work as agonists (Murphy et al. Genes that act downstream of DAF-16 to influence the lifespan of Caenorhabditis elegans. Nature 424, 277-283 (2003)), whereas others are antagonists (See, Li et al., daf-28 encodes a C. elegans insulin superfamily member that is regulated by environmental cues and acts in DAF-2 signaling pathway. Genes Dev. 17, 844-858 (2003)), to insulin signaling in worms indicates that glucose homeostasis could be more directly regulated by expression of insulin-like peptides in response to dietary restriction. In the future, it will be important to understand what part glucagon production plays in this process.

The response to IIS involves the DAF-16-dependent regulation of sod-1, sod-3 and sod-5, whereas dietary restriction involves the PHA-4-dependent expression of sod-1, sod-2, sod-4 and sod-5. The disparate transcriptional outcomes of these treatments on oxygen radical scavenging genes could suggest that a different form of reactive oxygen species production may be induced under conditions of reduced IIS than is induced under conditions of dietary restriction. This indicates divergent underlying metabolic consequences stemming from the manipulation of these independent pathways. Alternatively, as the expression patterns for most of these sods remain unknown, the differential transcriptional regulation of sods under IIS and dietary restriction could indicate distinct tissue-specific requirements for IIS and diet-restriction-mediated longevity. In C. elegans, IIS is required in the neurons and intestinal cells to regulate lifespan (Apfeld & Kenyon, Regulation of lifespan by sensory perception in Caenorhabditis elegans. Nature 402, 804-809 (1999); Libina et al., Tissue-specific activities of C. elegans DAF-16 in the regulation of lifespan. Cell 115, 489-502 (2003); Wolkow et al., Regulation of C. elegans lifespan by insulinlike signaling in the nervous system. Science 290, 147-150 (2000)). Although expression patterns of pha-4 overlap with those of daf-16 in the intestine and some neuronal cells, it is not known which tissues integrate and respond to reduced dietary intake. It is possible that the same tissues that exhibit increased levels of oxidative-stress response genes also will require DAF-16 or PHA-4 to affect longevity. It is likely that sod gene regulation is not the sole target of DAF-16 and PHA-4 for longevity assurance, but rather these transcription factors orchestrate a larger regulatory network that has been previously proposed (Murphy et al. Nature 424, 277-283 (2003); McElwee et al., Transcriptional outputs of the Caenorhabditis elegans forkhead protein DAF-16. Aging Cell 2, 111-121 (2003)).

Many of the physiological outcomes of animals with reduced IIS compared with animals undergoing dietary restriction are similar, including reduced body size, lower plasma IGF-1 and insulin levels, and increased insulin sensitivity. Furthermore, transcriptional profiling of long-lived dwarf mice, having reduced IGF-1 signaling, in combination with dietary restriction, additively increased expression of multiple liver-specific genes (Tsuchiya, T. et al. Additive regulation of hepatic gene expression by dwarfism and caloric restriction. Physiol. Genomics 17, 307-315 (2004)). However, compelling genetic analysis indicates that many key differences among IIS- and diet-restricted mice exist as well. For example, long-lived growth-hormone-deficient mice still respond to dietary restriction, and IGF-1R long lived heterozygous mice do not show protracted or reduced reproduction (Holzenberger, M. et al. IGF-1 receptor regulates lifespan and resistance to oxidative stress in mice. Nature 421, 182-187 (2003)). Therefore, given the discrepancy between the mode of action elicited by reduced IIS and dietary restriction that results in increased longevity of an organism, it is important to note that pha-4 is exceptionally specific for the longevity induced by dietary restriction. Reduction of pha-4 does not suppress the long lifespan of daf-2 mutant animals or animals with defective electron transport chains. Therefore, in agreement with previous reports (See, Houthoofd, K., et al. Life extension via dietary restriction is independent of the Ins/IGF1 signalling pathway in Caenorhabditis elegans. Exp. Gerontol. 38, 947-954 (2003); Lakowski & Hekimi. The genetics of caloric restriction in Caenorhabditis elegans. Proc. Natl. Acad. Sci. USA 95, 13091-13096 (1998)), the above examples show that there exists an independent pathway for the regulation of dietary restriction in worms. Consistent with this observation, worms undergoing dietary restriction do not require daf-16. Results instead suggest that dietary restriction impinges on an independent mechanism that ultimately increases the activity of PHA-4. Overexpression of pha-4 extends longevity in the absence of daf-16, and pha-4 expression is increased under conditions of dietary restriction. The above examples therefore provide the first findings of a forkhead transcription factor that acts independent of and with a parallel mechanism to daf-16 and IIS to regulate the ageing process in diet-restricted worms.

### Methods Summary

C. elegans strains, growth, imaging, lifespan analysis, Q-PCR and RNAi application were performed as previously described (Wolff, S. et al. SMK-1, an essential regulator of DAF-16-mediated longevity. Cell 124, 1039-1053 (2006)). For bacterial restriction studies, each lifespan consisted of 4 wells, with 1 ml of culture and 15 worms per well (n560). Lifespans were scored and worms transferred to new cultures every 3-4 days. Liquid cultures were prepared using an overnight culture of OP50 Escherichia coli grown at 37° C. Bacteria were washed three times in S-Basal medium. The bacterial concentration was adjusted to 1.5×10⁻⁹ cells ml⁻¹ in S-Basal medium containing cholesterol, carbenicillin, tetracycline and kanamycin. Serial dilutions were performed to achieve bacterial concentrations of 7.5×10⁸, 1.5×10⁸, 7.5×10⁷, 2.5×10⁷ and 5×10⁶ cells ml⁻¹. Cultures contained Fluorodeoxyuridine (FUDR) at 100 ugml⁻¹ for the first twelve days of lifespan analysis to block worm reproduction. For analysis of the temperature-sensitive pha-4(zu225) mutant allele, pha-4(zu225); smg-1(cc546ts)22 double mutant worms were grown at 25° C. to inactivate smg-1 and allow production of functional pha-4. pha-4 was inactivated by shifting double mutants to 15° C., restoring smg-1 activity, which results in degradation of the pha-4(zu225) allele after the first day of adulthood, thus avoiding any developmental defects owing to loss of pha-4 during larval stages. All control worms were treated identically.

While the foregoing invention has been described in some detail for purposes of clarity and understanding, it will be clear to one skilled in the art from a reading of this disclosure that various changes in form and detail can be made without departing from the true scope of the invention. For example, all the methods and screening systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method of screening for a modulator of longevity, the method comprising:
providing a non-human animal, which animal expresses pha-4 or a homolog thereof and exhibits reduced expression of daf-16 or a homolog thereof;
administering a test compound to the non-human animal; and,
assaying a pha-4 parameter in the non-human animal, wherein a change in the pha-4 parameter indicates the test compound modulates longevity.

2. The method of claim 1, wherein the pha-4 parameter comprises lifespan or an activity or expression level of pha-4, daf-16, sod-1, sod-2, sod-4, sod-5, foxa1, foxa2, foxa3, or any homologs thereof.

3. The method of claim 1, wherein the change in the pha-4 parameter comprises increased lifespan of the non-human animal or increased expression of pha-4, daf-16, sod-1, sod-2, sod-4, sod-5, foxa 1, foxa2, foxa3 or any homologs thereof.

4. The method of claim 1, wherein the non-human animal is a nematode.

5. The method of claim 4, wherein the nematode is C. elegans.

6. The method of claim 1, wherein the non-human animal is an adult.

7. The method of claim 1, wherein administering the modulator to the non-human animal comprises feeding the modulator to the non-human animal.

8. The method of claim 1, further comprising subjecting the non-human animal to dietary restriction.

9. The method of claim 1, wherein the non-human animal does not express daf-16.

10. A method for modulating longevity of an animal, the method comprising: modulating expression of pha-4 or a homolog thereof in the animal.

11. The method of claim 10, further comprising: modulating the expression of daf-16 or a homolog thereof in the animal.

12. The method of claim 10, wherein the method comprises increasing expression of pha-4 or the homolog thereof in the animal.

13. The method of claim 12, wherein the method further comprises decreasing expression of daf-16 or a homolog thereof in the animal.

14. The method of claim 10, wherein the method comprises administering a longevity modulator that affects pha-4 or the homolog thereof in the animal.

15. The method of claim 14, wherein the longevity modulator increases expression of pha-4 or the homolog thereof.

16. The method of claim 14, wherein the longevity modulator increases expression of pha-4 or the homolog thereof and decreases the expression of daf-16 or a homolog thereof.

17. The method of claim 10, further comprising subjecting the animal to dietary restriction.

18. The method of claim 10, wherein the homolog comprises a foxa gene.

19. A method of screening for a compound that modulates longevity, the method comprising:
contacting a cell that expresses pha-4 or a homolog thereof with a test agent, which cell also exhibits reduced expression of daf-16 or a homolog thereof; and,
assaying a pha-4 parameter in the cell, wherein a change in the pha-4 parameter relative to a control sample without the test agent identifies the compound that modulates longevity.

20. The method of claim 25, wherein the pha-4 parameter comprises an activity or expression level of pha-4, daf-16, sod-1, sod-2, sod-4, sod-5, foxa1, foxa2, foxa3, or any homologs thereof.

21. The method of claim 26, wherein the change in the expression level comprises an increase in expression.

22. A method of increasing longevity or delaying onset of an age-related disease in an animal, the method comprising administering to the animal a compound that increases expression of pha-4 or a homolog thereof.
